(19) United States
(12) Patent Application Publication
(10) Pub. No.: US 2026/0120891 A1
(43) Pub. Date: Apr. 30, 2026

(54) CONVERSATIONAL CHART SEARCH

(71) Applicant: Oracle International Corporation, Redwood Shores, CA (US)

(72) Inventors: Sneha Srinivasan, Bothell, WA (US); Aashna Devang Kanuga, Foster City, CA (US); Stephen Andrew McRitchie, Palo Alto, CA (US); Yuanxu Wu, Sunnyvale, CA (US); Yingqiong Shi, Redwood City, CA (US); Lezhi Wang, Dallas, TX (US)

(73) Assignee: Oracle International Corporation, Redwood Shores, CA (US)

(21) Appl. No.: 19/368,345

(22) Filed: Oct. 24, 2025

Related U.S. Application Data
(60) Provisional application No. 63/712,331, filed on Oct. 25, 2024.

(51) Int. Cl. G16H 70/60; G06F 16/242; G16H 50/70

(52) U.S. Cl. CPC G16H 70/60; G06F 16/243; G16H 50/70

(57) ABSTRACT

Agentic digital assistant methods and systems for generating a response to a user query are disclosed. A method includes receiving a query; identifying, from the input, one or more key phrases associated with one or more medical concepts; expanding the one or more medical concepts to include one or more other medical concepts that are related to the one or more medical concepts; identifying one or more portions of a database schema associated with the one or more medical concepts and the one or more other medical concepts; generating a prompt that comprises an instruction, the one or more portions of the database schema, and an utterance associated with the natural language component; transmitting the prompt to a machine learning model; receiving, from the machine learning model, a query result that includes information to answer the query; and providing the query result to the computing device associated with the user.

### CROSS-REFENCE TO RELATED APPLICATION

The present application claims the benefit of and priority to U.S. Provisional Application No. 63/712,331, filed on Oct. 25, 2024, the disclosure of which is incorporated herein by reference in its entirety for all purposes.

### TECHNICAL FIELD

The present disclosure relates generally to digital assistants, and, more particularly, to techniques for retrieving information used to answer a user's query.

### BACKGROUND

Artificial intelligence (AI) has diverse applications, with a notable evolution in the realm of digital assistants or chatbots. Originally, many users sought instant reactions through instant messaging or chat platforms. Organizations, recognizing the potential for engagement, utilized these platforms to interact with entities, such as end users, in real-time conversations.

However, maintaining alive communication channel with entities through human service personnel proved to be costly for organizations. In response to this challenge, digital assistants or chatbots, also known as bots, emerged as a solution to simulate conversations with entities, particularly over the Internet. The bots enabled entities to engage with users through messaging apps they already used or other applications with messaging capabilities.

Initially, traditional chatbots relied on predefined skill or intent models, which required entities to communicate within a fixed set of keywords or commands. Unfortunately, this approach limited the ability of the bot to engage intelligently and contextually in live conversations, hindering its capacity for natural communication. Entities were constrained by having to use specific commands that the bot could understand, often leading to difficulties in conveying intention effectively.

The landscape has since transformed with the integration of Large Language Models (LLMs). LLMs are deep learning algorithms that can perform a variety of natural language processing (NLP) tasks. They use a neural network architecture called a transformer, which can learn from the patterns and structures of natural language and conduct more nuanced and contextually aware conversations. This evolution marks a significant shift from rigid keyword-based interactions to a more adaptive and intuitive communication experience, enhancing the overall capabilities of digital assistants or chatbots in understanding and responding to user queries.

### BRIEF SUMMARY

Techniques disclosed herein pertain to a digital assistant configuration that provides increased accuracy. The digital assistant configuration employs a query understanding manager that expands medical concepts identified within a query to improve results generated from an LLM. The techniques described herein increase accuracy of the results and reduces latency due to the reduction in an amount of data provided to the LLM to generate the results.

In one aspect, a computer-implemented method includes receiving an input from an interface of a computing device associated with a user, the input comprising a query and a natural language component. The method may include identifying, from the input, one or more key phrases associated with one or more medical concepts, expanding the one or more medical concepts to include one or more other medical concepts that are related to the one or more medical concepts, identifying one or more portions of a database schema associated with the one or more medical concepts and the one or more other medical concepts, generating a prompt, wherein the prompt comprises an instruction, the one or more portions of the database schema, and an utterance associated with the natural language component, transmitting the prompt to a machine learning model, receiving, from the machine learning model, a query result that includes information to answer the query, and providing the query result to the computing device associated with the user.

Some embodiments include a system that includes one or more processing systems and one or more computer-readable media storing instructions which, when executed by the one or more processing systems, cause the system to perform part or all of the operations and/or methods disclosed herein.

Some embodiments include one or more non-transitory computer-readable media storing instructions which, when executed by one or more processing systems, cause a system to perform part or all of the operations and/or methods disclosed herein.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

FIG. 1 is a simplified block diagram of a distributed environment incorporating a digital assistant system in accordance with various embodiments.

FIG. 2 is an example of a user interface for configuring an agent of a digital assistant in accordance with various embodiments.

FIG. 3 is an example of a computing environment involving a digital assistant that can be implemented with generative artificial intelligence in accordance with various embodiments.

FIG. 4 is a flowchart of a process for generating a response to user input using a digital assistant that can be implemented using generative artificial intelligence in accordance with various embodiments.

FIG. 5 is a simplified block diagram of a distributed environment incorporating a clinical artificial intelligence agent system in accordance with various embodiments.

FIG. 6 is a simplified block diagram of another distributed environment incorporating a clinical artificial intelligence agent system in accordance with various embodiments.

FIG. 7 is a simplified block diagram of an approach for generating a response to a user's query, in accordance with various embodiments.

FIG. 8 is a block diagram of an approach for generating a response to a user's query, in accordance with various embodiments.

FIG. 9 shows an example flows for generating a response to a user's query, in accordance with various embodiments.

FIG. 10 is a block diagram illustrating one pattern for implementing a cloud infrastructure as a service system according to certain embodiments.

FIG. 11 is a block diagram illustrating another pattern for implementing a cloud infrastructure as a service system according to certain embodiments.

FIG. 12 is a block diagram illustrating another pattern for implementing a cloud infrastructure as a service system according to certain embodiments.

FIG. 13 is a block diagram illustrating another pattern for implementing a cloud infrastructure as a service system according to certain embodiments.

FIG. 14 is a block diagram illustrating an example computer system according to certain embodiments.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### Auentic Digital Assistant

Artificial intelligence techniques have broad applicability. For example, a digital assistant can be or include an artificial-intelligence-driven interface that helps users accomplish a variety of tasks using natural language conversations. For conventional digital assistants, such as those that do not involve generative artificial intelligence, a provider of the digital assistant may assemble one or more skills that can be focused on specific types of tasks such as tracking inventory, submitting timecards, and creating expense reports. When an end user engages with the digital assistant, the digital assistant can evaluate input provided by the end user to determine the intent of the end user and can route the conversation to and from the appropriate skill based on a perceived intent of the end user. However, there are some disadvantages of traditional intent-based skills including a limited understanding of natural language, inability to handle unknown inputs, limited ability to hold natural conversations off script, challenges integrating external knowledge, and the like.

User interactions with a digital assistant can lead to prompt responses to queries or the execution of requested actions. Additionally, these interactions have the potential to emulate human-like conversations, resembling a natural back-and-forth dialogue between a user and a human operator. To enhance user experience, digital assistants may also engage in multimodal communications, allowing users to convey information through spoken utterances or alternative input methods, such as selecting options on a computer display. However, achieving such functionalities efficiently with digital assistants, especially through natural language models, poses several challenges. For instance, understanding human speech remains a significant hurdle for natural language models, even those based on machine-learning. The scalability of the models can be problematic and inefficient, while their domain-specific limitations further complicate effective communication in various contexts.

The advent of generative artificial intelligence techniques and models, such as large language models (LLMs), has propelled the field of digital assistant design to unprecedented levels of sophistication and can be used to address the above and other technical problems associated with traditional intent-based skills. An LLM can be or include a neural network that employs a transformer architecture, which is specifically generated for processing and generating sequential data such as text or words in conversations. LLMs can undergo training with extensive textual data, and the training can gradually hone an ability to generate text that closely mimics human-written or spoken language.

Techniques are described herein to enhance LLMs with tools that empower or otherwise provide the LLMs access to external knowledge sources that provide the LLMs with the capability to recall facts and/or knowledge and facilitate the LLMs to better understand and respond to user queries in a contextually relevant manner. These tools, referred to herein as “agents,” provide the capability to recall facts and/or knowledge utilizing various techniques such as knowledge graphs, custom knowledge bases, Application Programming Interfaces (APIs), web crawling or scraping, and the like. In some examples, the tools, or the agents, can be powered or otherwise controlled by the LLMs. Once configured, the LLMs and agents can be deployed in artificial intelligence-based systems such as digital assistant applications. Users, such as end users or other entities, can interact with the digital assistant, such as by posing questions or making requests, and the LLMs and agents can work in tandem to generate responses based on a combination of a base LLM capability and access to the external knowledge via the agent. Using the LLMs and agents allows the digital assistant to provide more accurate, relevant, and contextually appropriate responses across a wide range of applications and domains.

For each digital assistant, a user (e.g., developer) may assemble LLMs and agents that interact to provide human-like conversation capabilities for various types of tasks such as tracking inventory, submitting timecards, updating accounts, creating expense reports, and the like. The LLMs are machine learning models trained on various tasks including plan creation using the LLM's in-context learning capability, knowledge or information retrieval on behalf of an agent, response generation to facilitate the human-like conversation, or any combination thereof. The agents are essentially containers having a software package containing everything needed to execute one or more actions defined for the agents. For example, the software package may include the code and any runtime configurations the code requires, application and system libraries, default values for any settings, and the like. The configuration parameters, settings, and customizations for dialog and routing/reasoning are primarily defined using natural language by a user (e.g., a developer). For example, users can provide configuration parameters that connect the agent to external assets, such as APIs, knowledge-based assets such as documents, URLs, LLMs, images, etc., data stores, prior conversations, etc. for executing one or more actions (e.g., change a user's 401k contribution). Once an agent is created, flow confirmation and testing may be performed through simulated conversations with LLMs and agents, and a digital assistant can then be implemented.

Implementation of an LLM-based digital assistant generally involves receiving a user input, such as a verbal request, command, or other statement (e.g., an utterance) from which the LLM digital assistant has a high-level awareness of the goal of the end user. A list of candidate agents is then determined based on the user input. The list of candidate agents includes agents configured to perform one or more actions that may potentially facilitate a response to the user input. Metadata for the agents in the list of candidate agents is then combined with the user input to construct an input prompt for an LLM. The LLM generates an execution plan that includes actions for facilitating a response to the user input based on the input prompt and metadata. The execution plan is then executed by an execution engine, which causes the agents to execute the actions. The actions may include internal task mapping in which a given action can be mapped to an API or semantic search knowledge task type. The execution of the actions generates output data from various sources, such as knowledge, API, SQL operations, etc., and/or relevant context and memory information from a context and memory store. The output data and relevant context and memory information are then combined with the user input to construct an output prompt for an LLM. The LLM synthesizes a response to the user input based on the output data and relevant context and memory information, and user input. The response is then sent to the user as an individual response or as part of a conversation with the user.

Advantageously, the LLM-based digital assistant described herein leverages reasoning capabilities of LLMs to drive decision-making and action orchestration to recall facts and/or knowledge and to facilitate the LLMs to better understand and respond to user queries in a contextually relevant manner. Additionally, or alternatively, the LLM-based digital assistant can eliminate a need for scripted dialog flows and provide out-of-the-box, human-like conversation capabilities.

A digital assistant can be or include a computer program that can perform conversations with end users. The digital assistant can generally respond to natural-language messages, such as questions and/or comments, through a messaging application (referred to herein as channels) that uses natural-language messages. The digital assistant can be made available to end users through a variety of channels, as well as via an application interface that may be developed to include a digital assistant, for example using a digital assistant software development kit. The channels may be or include an end-user-preferred messaging application that the end user has already installed and with which the end user may already be familiar. In some examples, the end user may not need to download and install new applications in order to converse with the digital assistant system. The channels may include, for example, over-the-top (OTT) messaging channels, such as Facebook™ Messenger, Facebook™ WhatsApp™, WeChat™, Line, Kik™, Telegram™, Talk, Skype™ Slack™, or SMS), virtual private assistants (such as Amazon™ Dot, Echo, or Show, Google™ Home, Apple™ HomePod™, etc.), mobile and web app extensions that extend native or hybrid/responsive mobile apps or web applications with chat capabilities, or voice based input such as devices or apps with interfaces that use Siri™, Cortana™, Google™ Voice, or other speech input for interaction.

The channels can carry the chat back and forth from end users to the digital assistant and various LLMs associated with the digital assistant. During the back-and-forth exchanges, the LLMs can receive the processed input in the form of a query and can process the query to generate a response. An LLM can predict the most contextually relevant and grammatically correct response based on training data used to train the LLM and based on received input such as the query and actions executed by the agents. The generated response may undergo post-processing to ensure adherence to guidelines, policies, and formatting standards associated with the digital assistant. This post-processed response may be more coherent and user-friendly than other responses that do not undergo post-processing. The post-processed response can be delivered to the user through the appropriate channel, which may be or include a text-based chat interface, a voice-based system, or another medium. According to various embodiments, the digital assistant can maintain the conversation context, allowing for further interactions and dynamic back-and-forth exchanges between the user and the LLMs where later interactions can build upon earlier interactions.

In some embodiments, the digital assistant system may intelligently handle end user interactions without interaction with a provider, such as an administrator or developer, of the digital assistant system. For example, an end user may send one or more messages to the digital assistant system in order to achieve a desired goal. A message may include certain content, such as text, emojis, audio, image, video, or other method of conveying a message. In some embodiments, the digital assistant system may convert the content into a standardized form, such as a representational state transfer (REST) or API call, against enterprise services with the proper parameters, and generate a natural language response. The digital assistant system may also prompt the end user for additional input parameters or request other additional information. In some embodiments, the digital assistant system may also initiate communication with the end user, rather than passively responding to end user utterances. Various techniques can be used for identifying an explicit invocation of a digital assistant system and determining an input for the digital assistant system being invoked. In certain embodiments, explicit invocation analysis can be performed by a master digital assistant based at least in part on detecting an invocation name in an utterance. In response to detecting the invocation name, the utterance may be refined or pre-processed for input to a digital assistant that is identified to be associated with the invocation name and/or communication.

### Medical Concept Expansion

Clinicians often face challenges in retrieving relevant information quickly due to the complexity of Electronic Health Record (EHR) systems and the structured nature of the data. In some examples, a data agent provides a more efficient and intuitive access to structured patient data within Electronic Health Records (EHRs) compared to prior systems. In some examples, the data agent receives natural language queries through a conversational interface powered by a Semantic Object Model (SOM) schema.

Generally, a Semantic Object Model (SOM) schema is a framework for representing information with relationships. In some examples, the SOM schema is for medical data that represents healthcare information with meaningful, unambiguous relationships. By defining the meaning and context of data, it enables interoperability between different systems, reduces data errors, and supports advanced analytics in healthcare.

In some examples, the data agent receives a query from a user from an interface of a computing device associated with the user. For instance, a natural language query (e.g., “Has she gotten any imaging done?”, “Has she had a urine analysis yet?”, “What sensitivities does she have in her cultures?”, . . . ) may be received.

After receiving the query, a query understanding manager identifies the medical concepts that are included within the query. In some cases, the key phrases/entities associated with one or more medical concepts are identified within the query. As used herein, a “medical concept” may represent a unique clinical meaning, such as a disease, diagnosis, procedure, or anatomical structure, represented by a unique code and human-readable descriptions. In some examples, the medical concepts can be organized into hierarchies (e.g., from general to more specific). According to some configurations, the medical concepts can be represented by a medical coding system such as one or more of Systematized Nomenclature of Medicine—Clinical Terms (SNOMED CT), Prescription Normalization (RxNORM), Logical Observation Identifiers Names and Codes (LOINC), International Classification of Diseases (ICD-10), and the like.

In some examples, the identified medical concepts are expanded to include one or more other medical concepts that are related to the identified medical concepts. According to some configurations, a search of a vector database containing embeddings of medical concepts (including medical codes and other relevant information) of one or more medical coding systems (e.g., SNOMED-CT, RxNORM, . . . ) is performed to identify medical concepts that are similar to the medical concepts identified in the query.

According to some configurations, the similarity search may use an embeddings-based Approximate Nearest Neighbor (ANN) search algorithm, which is a search approach that can use various similarity metrics, including cosine similarity (as a distance metric). Those skilled in the art will appreciate various ways and techniques that can be used to perform similarity searches. The techniques may include, but are not limited to, ANN search algorithm, Locality-sensitive hashing (LSH), K-nearest neighbors (KNN), and the like.

In some examples, the search returns a ranked list of medical concepts based on the identified medical concepts and their associated similarity scores. At least a portion of the medical concepts are selected from the ranked list according to certain criteria (e.g., top ten of all medical concepts in the list, top two from each medical entity type in the ranked list, within a specified range of a similarity score, . . . ) to expand the medical concepts.

In some examples, a Fast Healthcare Interoperability Resources (FHIR)-compliance data structure can be generated that helps ensure consistency in the recording and reporting medical information and Interoperability among platforms. FHIR may refer to a standard for exchanging healthcare information electronically between different systems, which may utilize modern web technologies such as RESTful APIs and JSON/XML format to facilitate the exchange. By identifying the related medical concepts, the scope of the search can be broadened without also having to search unrelated medical concepts.

After identifying the medical concepts within the query and other medical concepts that are similar, one or more portions of a schema associated with the medical concepts are identified. Generally, the portions of the schema associated with the identified medical are obtained. In some configurations, the query understanding manager combines the portions of a database schema that represents the identified medical concepts within the query and the portions of the database schema that represents the medical concepts identified to be similar.

In some examples, a prompt (that is provided to a machine learning model) is generated that comprises an instruction, the portions of the database schema associated with the identified medical concepts, and at least a portion of the received user input received (e.g., an utterance associated with a natural language component of the query). In some configurations, generating the prompt can include providing the one or more portions of the database schema to a second machine learning model to obtain structured object query language that can be used to obtain information from one or more databases. The generated prompt can then be provided to an execution engine 708 to execute the associated execution plan 706 that generates a response to the query. In some examples, the generated prompt includes SOQL query language generated by a data action planner LLM used by the query understanding manager. The query result can then be provided to the user who made the query.

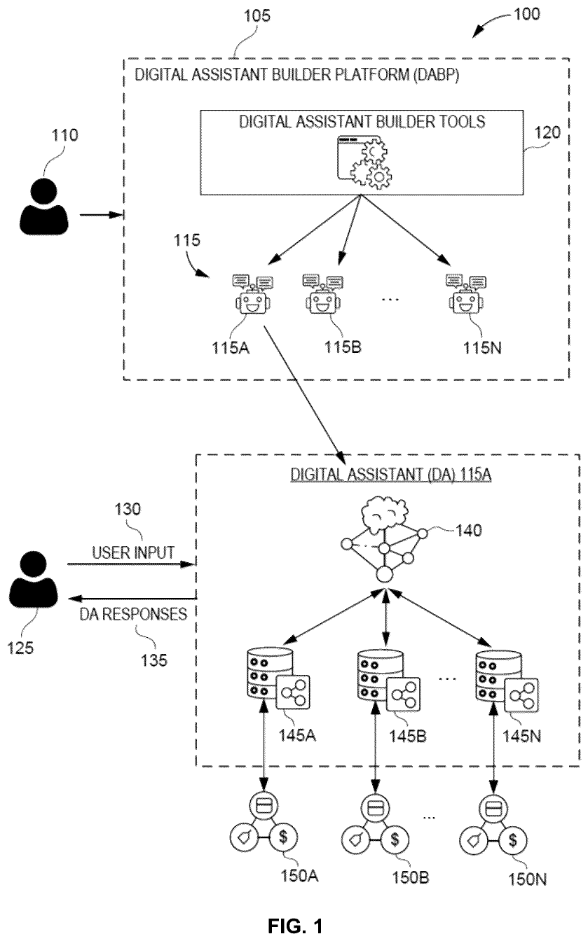

FIG. 1 is a simplified block diagram of an environment 100 incorporating a digital assistant system according to certain embodiments. Environment 100 includes a digital assistant builder platform (DABP) 105 that enables users 110 to create and deploy digital assistant systems 115. For purposes of this disclosure, a “digital assistant” is an entity that helps users of the digital assistant accomplish various tasks through natural language conversations. A digital assistant can be implemented using software only (e.g., the digital assistant is a digital entity implemented using programs, code, or instructions executable by one or more processors), using hardware, or using a combination of hardware and software. A digital assistant can be embodied or implemented in various physical systems or devices, such as in a computer, a mobile phone, a watch, an appliance, a vehicle, and the like. A digital assistant is also sometimes referred to as a chatbot system. Accordingly, for purposes of this disclosure, the terms digital assistant and chatbot system are interchangeable.

DABP 105 can be used to create one or more digital assistants (or DAs) systems. For example, as illustrated in FIG. 1, user 110 representing a particular enterprise can use DABP 105 to create and deploy a digital assistant 115A for users of the particular enterprise. For example, DABP 105 can be used by a bank to create one or more digital assistants for use by the bank's customers, for example to change a 401k contribution, etc. The same DABP 105 platform can be used by multiple enterprises to create digital assistants. As another example, an owner of a restaurant, such as a pizza shop, may use DABP 105 to create and deploy digital assistant 115B that enables customers of the restaurant to order food (e.g., order pizza).

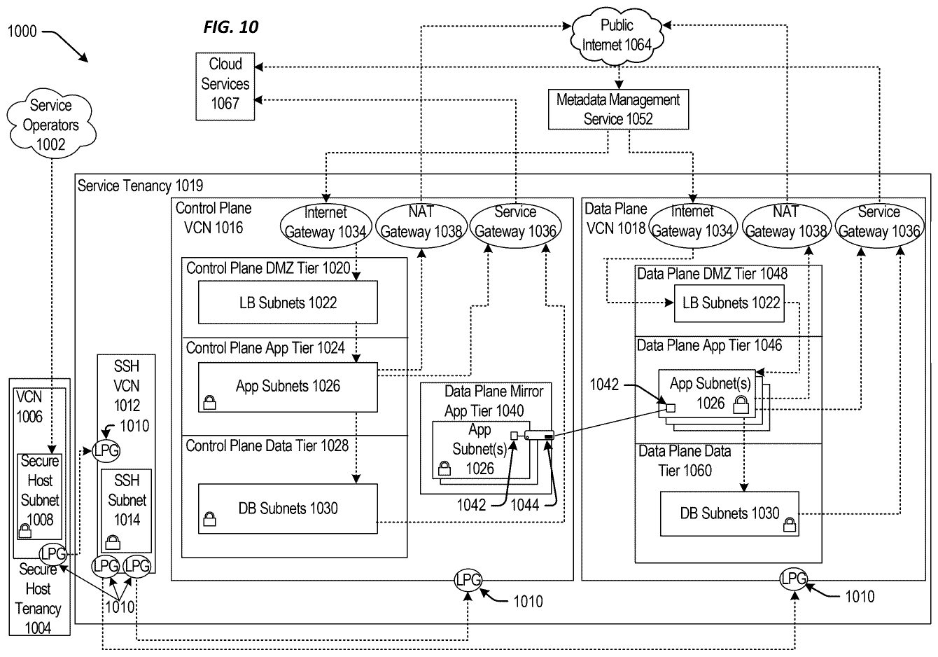

To create one or more digital assistant systems 115, the DABP 105 is equipped with a suite of tools 120, enabling the acquisition of LLMs, agent creation, asset identification, and deployment of digital assistant systems within a service architecture (described herein in detail with respect to FIG. 1) for users via a computing platform such as a cloud computing platform described in detail with respect to FIGS. 10-14. In some instances, the tools 120 can be utilized to access pre-trained and/or fine-tuned LLMs from data repositories or computing systems. The pre-trained LLMs serve as foundational elements, possessing extensive language understanding derived from vast datasets. This capability enables the models to generate coherent responses across various topics, facilitating transfer learning. Pre-trained models offer cost-effectiveness and flexibility, which allows for scalable improvements and continuous pre-training with new data, often establishing benchmarks in Natural Language Processing (NLP) tasks. Conversely, fine-tuned models are specifically trained for tasks or industries (e.g., plan creation utilizing the LLM's in-context learning capability, knowledge or information retrieval on behalf of an agent, response generation for human-like conversation, etc.), enhancing their performance on specific applications and enabling efficient learning from smaller, specialized datasets. Fine-tuning provides advantages such as task specialization, data efficiency, quicker training times, model customization, and resource efficiency. In some embodiments, fine-tuning may be particularly advantageous for niche applications and ongoing enhancement.

In other instances, the tools 120 can be utilized to pre-train and/or fine-tune the LLMs. The tools 120, or any subset thereof, may be standalone or part of a machine-learning operationalization framework, inclusive of hardware components like processors (e.g., CPU, GPU, TPU, FPGA, or any combination), memory, and storage. This framework operates software or computer program instructions (e.g., TensorFlow, PyTorch, Keras, etc.) to execute arithmetic, logic, input/output commands for training, validating, and deploying machine-learning models in a production environment. In certain instances, the tools 120 implement the training, validating, and deploying of the models using a cloud platform such as Oracle Cloud Infrastructure (OCI). Leveraging a cloud platform can make machine-learning more accessible, flexible, and cost-effective, which can facilitate faster model development and deployment for developers.

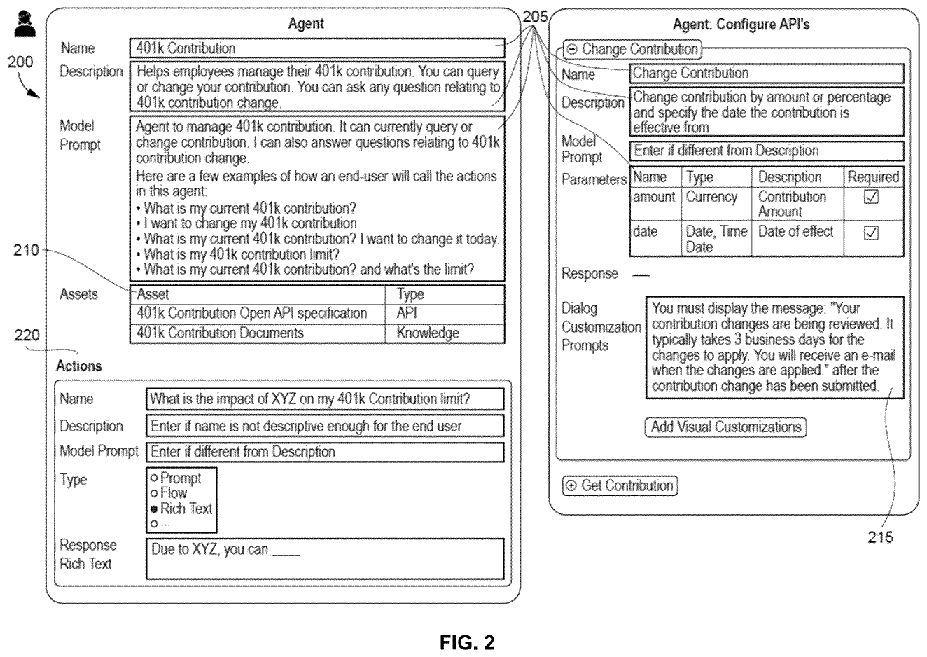

The tools 120 further include a prompt-based agent composition unit for creating agents and their associated actions (e.g., a prompt such as Tell me a joke, implicit Change Contribution, and Get Contribution API calls) that an end-user can end up invoking. As shown in FIG. 2, the agents (e.g., 401k Change Contribution Agent 200) are primarily defined as a compilation of agent artifacts 205 using natural language within the prompt-based agent composition unit. Users 110 can create functional agents quickly by providing agent artifact 205 information, parameters, and configurations and by pointing to assets 210. The assets 210 are resources such as APIs for interfacing with applications, files and/or documents for retrieving knowledge, data stores for interacting with data, and the like available to the agents for the execution of actions. The assets 210 are imported, and then the users 110 can use natural language again to provide additional API customizations for dialog and routing/reasoning 215. Most of what an agent does involves executing actions 220. An action 220 can be an explicit one that's authored using natural language (similar to creating agent artifacts—e.g., ‘What is the impact of XYZ on my 401k Contribution limit?’ action in the below ‘401k Contribution Agent’ figure) or an implicit one that is created when an asset is imported (automatically imported upon pointing to a given asset based on metadata and/or specifications associated with the asset—e.g., actions created for Change Contribution and Get Contribution API in the below ‘401k Contribution Agent’ figure). In the agent example illustrated in FIG. 2, the design time user can easily create explicit actions. For example, the user chooses the ‘Rich Text’ action type (see Table 1 for a list of exemplary action types) and creates the name artifact ‘What is the impact of XYZ on my 401k Contribution limit?’ when the user learns that a new FAQ needs to be added, as it's not currently in the knowledge documents (assets 210) the agent references (thus was not implicitly added as an action).

TABLE 1

| | Action | |
|---|---|---|
| | Type | Description |
| 1 | Prompt | The action is implemented using a prompt to an LLM. |
| 2 | Rich | The action is implemented using rich text. The most |
| | Text | common use case is FAQs. |
| 3 | Flow | The action is implemented using Visual Flow Designer |
| | | flow. May be used for complex cases where the developer |
| | | is not able to use the out-of-the-box dialogue and dialog |
| | | customizations. |

There are various ways in which the agents and assets can be associated or added to a digital assistant 115. In some instances, the agents can be developed by an enterprise and then added to a digital assistant using DABP 105. In other instances, the agents can be developed and created using DABP 105 and then added to a digital assistant created using DABP 105. In yet other instances, DABP 105 provides an online digital store (referred to as an “agent store”) that offers various pre-created agents directed to a wide range of tasks and actions. The agents offered through the agent store may also expose various cloud services. In order to add the agents to a digital assistant being generated using DABP 105, a user 110 of DABP 105 can access assets via tools 120, select specific assets for an agent, initiate a few mock chat conversations with the agent, and indicate that the agent is to be added to the digital assistant created using DABP 105.

Once deployed in a production environment, such as the architecture described with respect to FIG. 1, a digital assistant, such as digital assistant 115A built using DABP 105, can be used to perform various tasks via natural language-based conversations between the digital assistant 115A and its users 125. As described above, the digital assistant 115A illustrated in FIG. 1 can be made available or accessible to its users 125 through a variety of different channels, such as but not limited to, via certain applications, via social media platforms, via various messaging services and applications, and other applications or channels. A single digital assistant can have several channels configured for it so that it can be run on and be accessed by different services simultaneously.

As part of a conversation, a user 125 may provide one or more user inputs 130 to digital assistant 115A and get responses 135 back from digital assistant 115A. A conversation can include one or more user inputs 130 and responses 135. Via these conversations, a user 125 can request one or more tasks to be performed by the digital assistant 115A and, in response, the digital assistant 115A is configured to perform the user-requested tasks and respond with appropriate responses to the user 125 using one or more LLMs 140.

User inputs 130 are generally in a natural language form and are referred to as utterances, which may also be referred to as prompts, queries, requests, and the like. The user inputs 130 can be in text form, such as when a user types in a sentence, a question, a text fragment, or even a single word and provides it as input to digital assistant 115A. In some embodiments, a user input 130 can be in audio input or speech form, such as when a user says or speaks something that is provided as input to digital assistant 115A. The user inputs 130 are typically in a language spoken by the user 125. For example, the user inputs 130 may be in English, or some other language. When a user inputs 130 is in speech form, the speech input is converted to text form user inputs 130 in that particular language and the text utterances are then processed by digital assistant 115A. Various speech-to-text processing techniques may be used to convert a speech or audio input to a text utterance, which is then processed by digital assistant 115A. In some embodiments, the speech-to-text conversion may be done by digital assistant 115A itself. For purposes of this disclosure, it is assumed that the user inputs 130 are text utterances that have been provided directly by a user 125 of digital assistant 115A or are the results of conversion of input speech utterances to text form. This, however, is not intended to be limiting or restrictive in any manner.

The user inputs 130 can be used by the digital assistant 115A to determine a list of candidate agents 145A-N. The list of candidate agents (e.g., 145A-N) includes agents configured to perform one or more actions that could potentially facilitate a response 135 to the user input 130. The list may be determined by running a search, such as a semantic search, on a context and memory store that has one or more indices comprising metadata for all agents 145 available to the digital assistant 115A. Metadata for the candidate agents 145A-N in the list of candidate agents is then combined with the user input to construct an input prompt for the one or more LLMs 140.

Digital assistant 115A is configured to use one or more LLMs 140 to apply NLP techniques to text and/or speech to understand the input prompt and apply natural language understanding (NLU) including syntactic and semantic analysis of the text and/or speech to determine the meaning of the user inputs 130. Determining the meaning of the utterance may involve identifying the goal of the user, one or more intents of the user, the context surrounding various words or phrases or sentences, one or more entities corresponding to the utterance, and the like. The NLU processing can include parsing the received user inputs 130 to understand the structure and meaning of the utterance, refining and reforming the utterance to develop a better understandable form (e.g., logical form) or structure for the utterance. The NLU processing performed can include various NLP-related processing such as sentence parsing (e.g., tokenizing, lemmatizing, identifying part-of-speech tags for the sentence, identifying named entities in the sentence, generating dependency trees to represent the sentence structure, splitting a sentence into clauses, analyzing individual clauses, resolving anaphoras, performing chunking, and the like). In certain instances, the NLU processing, or any portions thereof, is performed by the LLMs 140 themselves. In other instances, the LLMs 140 use other resources to perform portions of the NLU processing. For example, the syntax and structure of an input utterance sentence may be identified by processing the sentence using a parser, a part-of-speech tagger, a named entity recognition model, a pretrained language model such as BERT, or the like.

Upon understanding the meaning of an utterance, the one or more LLMs 140 generate an execution plan that identifies one or more agents (e.g., agent 145A) from the list of candidate agents to execute and perform one or more actions or operations responsive to the understood meaning or goal of the user. The one or more actions or operations are then executed by the digital assistant 115A on one or more assets (e.g., asset 150A—knowledge, API, SQL operations, etc.) and/or the context and memory store. The execution of the one or more actions or operations generates output data from one or more assets and/or relevant context and memory information from a context and memory store comprising context for a present conversation with the digital assistant 115A. The output data and relevant context and memory information are then combined with the user input 130 to construct an output prompt for one or more LLMs 140. The LLMs 140 synthesize the response 135 to the user input 130 based on the output data and relevant context and memory information, and the user input 130. The response 135 is then sent to the user 125 as an individual response or as part of a conversation with the user 125.

For example, a user input 130 may request a pizza to be ordered by providing an utterance such as “I want to order a pizza.” Upon receiving such an utterance, digital assistant 115A is configured to understand the meaning or goal of the utterance and take appropriate actions. The appropriate actions may involve, for example, providing responses 135 to the user with questions requesting user input on the type of pizza the user desires to order, the size of the pizza, any toppings for the pizza, and the like. The questions requesting user may be generated by executing an action via an agent (e.g., agent 145A) on a knowledge asset (e.g., a menu for a pizza restaurant) to retrieve information that is pertinent to ordering a pizza (e.g., to order a pizza a user must provide type, seize, topping, etc.). The responses 135 provided by digital assistant 115A may also be in natural language form and typically in the same language as the user input 130. As part of generating these responses 135, digital assistant 115A may perform natural language generation (NLG) using the one or more LLMs 140. For the user ordering a pizza, via the conversation between the user and digital assistant 115A, the digital assistant 115A may guide the user to provide all the requisite information for the pizza order, and then at the end of the conversation cause the pizza to be ordered. The ordering may be performed by executing an action via an agent (e.g., agent 145A) on an API asset (e.g., an API for ordering pizza) to upload or provide the pizza order to the ordering system of the restaurant. Digital assistant 115A may end the conversation by generating a final response 135 providing information to the user 125 indicating that the pizza has been ordered.

While the various examples provided in this disclosure describe and/or illustrate utterances in the English language, this is meant only as an example. In certain embodiments, digital assistants 115 are also capable of handling utterances in languages other than English. Digital assistants 115 may provide subsystems (e.g., components implementing NLU functionality) that are configured for performing processing for different languages. These subsystems may be implemented as pluggable units that can be called using service calls from an NLU core server. This makes the NLU processing flexible and extensible for each language, including allowing different orders of processing. A language pack may be provided for individual languages, where a language pack can register a list of subsystems that can be served from the NLU core server.

While the embodiment in FIG. 1 illustrates the digital assistant 115A including one or more LLMs 140 and one or more agents 145A-N, this is not intended to be limiting. A digital assistant can include various other components (e.g., other systems and subsystems as described in greater detail with respect to FIG. 1) that provide the functionalities of the digital assistant. The digital assistant 115A and its systems and subsystems may be implemented only in software (e.g., code, instructions stored on a computer-readable medium and executable by one or more processors), in hardware only, or in implementations that use a combination of software and hardware.

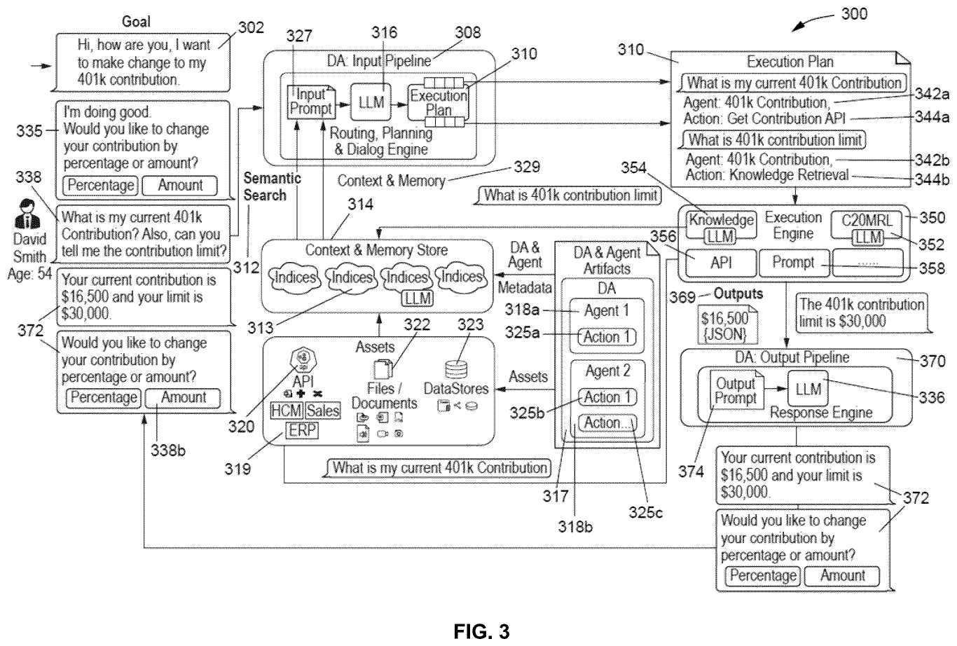

FIG. 3 is an example of an architecture for a computing environment 300 for a digital assistant implemented with generative artificial intelligence in accordance with various embodiments. As illustrated in FIG. 3, an infrastructure and various services and features can be used to enable a user to interact with a digital assistant (e.g., digital assistant 115A described with respect to FIG. 1) based at least in part on a series of prompts such as a conversation. The following is a detailed walkthrough of a conversation flow and the role and responsibility of the components, services, models, and the like of the computing environment 300 within the conversation flow. In this walkthrough, it is assumed that a user “David” is interested in making a change to his 401k contribution, and in an utterance 302, David provides the following input to the digital assistant: Hi, how are you, I want to make a change to my 401k contribution.

The utterance 302 can be communicated to the digital assistant (e.g., via text dialogue box or microphone) and provided as input to the input pipeline 308. The input pipeline 308 is used by the digital assistant to create an execution plan 310 that identifies one or more agents to address the request in the utterance 302 and one or more actions for the one or more agents to execute for responding to the request. A two-step approach can be taken via the input pipeline 308 to generate the execution plan 310. First, a search 312 can be performed to identify a list of candidate agents. The search 312 comprises running a query on indices 313 of a context and memory store 314 based on the utterance 302. In some instances, the search 312 is a semantic search performed using words from the utterance 302, The semantic search uses NLP and optionally machine learning techniques to understand the meaning of the utterance 302 and retrieve relevant information from the context and memory store 314. In contrast to traditional keyword-based searches, which rely on exact matches between the words in the query and the data in the context and memory store 314, a semantic search takes into account the relationships between words, the context of the query, synonyms, and other linguistic nuances. This allows the digital assistant to provide more accurate and contextually relevant results, making it more effective in understanding the user's intent in the utterance 302.

The context and memory store 314 is implemented using a data framework for connecting external data to LLMs 316 to make it easy for users to plug in custom data sources. The data framework provides rich and efficient retrieval mechanisms over data from various sources such as files, documents, datastores, APIs, and the like. The data can be external (e.g., enterprise assets) and/or internal (e.g., user preferences, memory, digital assistant, and agent metadata, etc.). In some instances, the data comprises metadata extracted from artifacts 317 associated with the digital assistant and its agents 318 (e.g., 318a and 318b). The artifacts 317 for the digital assistant include information on the general capabilities of the digital assistant and specific information concerning the capabilities of each of the agents 318 (e.g., actions 220 described with respect to FIG. 2) available to the digital assistant (e.g., agent artifacts 205 described with respect to FIG. 2). Additionally, or alternatively, the artifacts 317 can encompass parameters or information associated with the artifacts 317 and that can be used to define the agents 318 in which the parameters or information associated with the artifacts 317 can include a name, a description, one or more actions, one or more assets, one or more customizations, etc. In some instances, the data further includes metadata extracted from assets 319 associated with the digital assistant and its agents 318 (e.g., 318a and 318b). The assets 319 may be resources, such as APIs 320, files and/or documents 322, data stores 323, and the like, available to the agents 318 for the execution of actions (e.g., actions 325a, 325b, and 325c). The data is indexed in the context and memory store 314 as indices 313, which are data structures that provide a fast and efficient way to look up and retrieve specific data records within the data. Consequently, the context and memory store 314 provides a searchable comprehensive record of the capabilities of all agents and associated assets that are available to the digital assistant for responding to the request.

The results of the search 312 include a list of candidate agents that are not just available to the digital assistant for responding to the request but also potentially capable of facilitating the generation of a response to the utterance 302. The list of candidate agents includes the metadata (e.g., metadata extracted from artifacts 317 and assets 319) from the context and memory store 314 that is associated with each of the candidate agents. The list can be limited to a predetermined number of candidate agents (e.g., top 10) that satisfy the query or can include all agents that satisfy the query. The list of candidate agents with associated metadata is appended to the utterance 302 to construct an input prompt 327 for the LLM 316. In some instances, context 329 concerning the utterance 302 are additionally appended to the list of candidate agents and the utterance 302. The context 329 is retrievable from the context and memory store 314 and includes user session information, dialog state, conversation or contextual history, user information, or any combination thereof. The search 312 is important to the digital assistant because it filters out agents that are unlikely to be capable of facilitating the generation of a response to the utterance 302. This filter ensures that the number of tokens (e.g., word tokens) generated from the input prompt 327 remains under a maximum token limit or context limit set for the LLM 316. Token limits represent the maximum amount of text that can be inputted into an LLM. This limit is of a technical nature and arises due to computational constraints, such as memory and processing resources, and thus makes certain that the LLMs are capable of taking the input prompt as input.

The second step of the two-step approach is for the LLM 316 to generate an execution plan 310 based on the input prompt 327. The LLM 316 has a deep generative model architecture (e.g., a reversible or autoregressive architecture with) for generating the execution plan 310. In some instances, the LLM 316 has over 100 billion parameters and generates the execution plan 310 using autoregressive language modeling within a transformer architecture, allowing the LLM 316 to capture complex patterns and dependencies in the input prompt 327. The LLM's 316 ability to generate the execution plan 310 is a result of its training on diverse and extensive textual data, enabling the LLM to understand human language across a wide range of contexts. During training, the LLM 316 learns to predict the next word in a sequence given the context of the preceding words. This process involves adjusting the model's parameters (weights and biases) based on the errors between its predictions and the actual next words in the training data. When the LLM 316 receives an input such as the input prompt 327, the LLM 316 tokenizes the text into smaller units such as words or sub-words. Each token is then represented as a vector in a high-dimensional space. The LLM 316 processes the input sequence token by token, maintaining an internal representation of context. The LLM's 316 attention mechanism allows it to weigh the importance of different tokens in the context of generating the next word. For each token in the vocabulary, the LLM 316 calculates a probability distribution based on its learned parameters. This probability distribution represents the likelihood of each token being the next word given the context. To generate the execution plan 310, the LLM 316 samples a token from the calculated probability distribution. The sampled token becomes the next word in the generated sequence. This process is repeated iteratively, with each newly generated token influencing the context for generating the subsequent token. The LLM 316 can continue generating tokens until a predefined length or stopping condition is reached.

In some instances, as illustrated in FIG. 3, the LLM 316 may not be able to generate a complete execution plan 310 because it is missing information such as if more information is required to determine an appropriate agent for the response, execute one or more actions, or the like. In this particular instance, the LLM 316 has determined that in order to change the 401k contribution as request by the user, it is necessary to understand whether the user would like to change the contribution by a percentage or certain currency amount. In order to obtain this information, the LLM 316 (or another LLM such as LLM 336) generates end-user response 335 (I'm doing good. Would you like to change your contribution by percentage or amount?[Percentage][Amount]) to the input prompt 327 that can obtain the missing information such that the LLM 316 is able to generate a complete execution plan 310. In some instances, the response may be rendered within a dialogue box of a GUI having one or more GUI elements allowing for an easier response by the user. In other instances, the response may be rendered within a dialogue box of a GUI allowing for the user to reply using the dialogue box (or alternative means such as a microphone). In this particular instance, the user responds with an additional query 338 (What is my current 401k Contribution?Also, can you tell me the contribution limit?) to gather additional information such that the user can reply to the response 335. The subsequent response—additional query 338—is input into the input pipeline 308 and the same processes described above with respect to utterance 302 are executed but this time with the context of the prior utterances/replies (e.g., utterance 302 and response 335) from the user's conversation with the digital assistant. This time, as illustrated in FIG. 3, the LLM 316 is able to generate a complete execution plan 310 because it has all the information it needs.

The execution plan 310 includes an ordered list of agents and/or actions that can be used and/or executed to sufficiently respond to the request such as the additional query 338. For example, and as illustrated in FIG. 3, the execution plan 310 can be an ordered list that includes a first agent 342a capable of executing a first action 344a via an associated asset and a second agent 342b capable of executing a second action 344b via an associated asset. The agents, and by extension the actions, may be ordered to cause the first action 344a to be executed by the first agent 342a prior to causing the second action 344b to be executed by the second agent 342b. In some instances, the execution plan 310 may be ordered based on dependencies indicated by the agents and/or actions included in the execution plan 310. For example, if executing the second agent 342b is dependent on, or otherwise requires, an output generated by the first agent 342a executing the first action 344a, then the execution plan 310 may order the first agent 342a and the second agent 342b to comply with the dependency. As should be understood, other examples of dependencies are possible.

The execution plan 310 is then transmitted to an execution engine 350 for implementation. The execution engine 350 includes a number of engines, including a natural language-to-programming language translator 352, a knowledge engine 354, an API engine 356, a prompt engine 358, and the like. for executing the actions of agents and implementing the execution plan 310. For example, the natural language-to-programming language translator 352, such as a Conversation to Oracle Meaning Representation Language (C2OMRL) model, may be used by an agent to translate natural language into a intermedial logical for (e.g., OMRL), convert the intermediate logical form into a system programming language (e.g., SQL) and execute the system programming language (e.g., execute an SQL query) on an asset 319 such as data stores 323 to execute actions and/or obtain data or information. The knowledge engine 354 may be used by an agent to obtain data or information from the context and memory store 314 or an asset 319 such as files/documents 322. The API engine 356 may be used by an agent to call an API 320 and interface with an application such as retirement fund account management application to execute actions and/or obtain data or information. The prompt engine 358 may be used by an agent to construct a prompt for input into an LLM such as an LLM in the context and memory store 314 or an asset 319 to execute actions and/or obtain data or information.

The execution engine 350 implements the execution plan 310 by running each agent and executing each action in order based on the ordered list of agents and/or actions using the appropriate engine(s). To facilitate this implementation, the execution engine 350 is communicatively connected (e.g., via a public and/or provue network) with the agents (e.g., 342a, 342b, etc.), the context and memory store 314, and the assets 319. For example, as illustrated in FIG. 3, when the execution engine 350 implements the execution plan 310, it will first execute the agent 342a and action 344a using API engine 356 to call the API 320 and interface with a retirement fund account management application to retrieve the user's current 401k contribution. Subsequently, the execution engine 350 can execute the agent 342b and action 344b using knowledge engine 354 to retrieve knowledge on 401k contribution limits. In some instances, the knowledge is retrieved by knowledge engine 354 from the assets 319 (e.g., files/documents 322). In other instances (as in this particular instance), the knowledge is retrieved by knowledge engine 354 from the context and memory store 314. Knowledge retrieval and action execution using the context and memory store 314 may be implemented using various techniques including internal task mapping and/or machine learning models such as additional LLM models. For example, the query and associated agent for “What is 401k contribution limit” may be mapped to a ‘semantic search’ knowledge task type for searching the indices 313 within the context and memory store 314 for a response to a given query. By way of another example, a request such as “Can you summarize the key points relating to 401k contribution” can be or include a ‘summary’ knowledge task type that may be mapped to a different index within the context and memory store 314 having an LLM trained to create a natural language response (e.g., summary of key points relating to 401k contribution) to a given query. Over time, a library of generic end-user task or action types (e.g., semantic search, summarization, compare/contrast, heterogeneous data synthesis, etc.) may be built to ensure that the indices and models within the context and memory store 314 are optimized to the various task or action types.

The result of implementing the execution plan 310 is output data 369 (e.g., results of actions, data, information, etc.), which is transmitted to an output pipeline 370 (also referred to herein as response engine 370) for generating end-user responses 372. For example, the output data 369 from the assets 319 (knowledge, API, dialog history, etc.) and relevant information from the context and memory store 314 can be transmitted to the output pipeline 370. The output data 369 is appended to the utterance 302 to construct an output prompt 374 for input to the LLM 336. In some instances, context 329 concerning the utterance 302 are additionally appended to the output data 369 and the utterance 302. The context 329 is retrievable from the context and memory store 314 and includes user session information, dialog state, conversation or contextual history, user information, or any combination thereof. The LLM 336 generates responses 372 based on the output prompt 374. In some instances, the LLM 336 is the same or similar model as LLM 316. In other instances, the LLM 336 is different from LLM 316 (e.g., trained on a different set of data, a different architecture, trained for a one or more different tasks, etc.). In either instance, the LLM 336 has a deep generative model architecture (e.g., a reversible or autoregressive architecture with) for generating the responses 372 using similar training and generative processes described above with respect to LLM 316. In some instances, the LLM 336 has over 100 billion parameters and generates the responses 372 using autoregressive language modeling within a transformer architecture, allowing the LLM 336 to capture complex patterns and dependencies in the output prompt 374.

In some instances, the end-user responses 372 may be in the format of a Conversation Message Model (CMM) and output as rich multi-modal responses. The CMM defines the various message types that the digital assistant can send to the user (outbound), and the user can send to the digital assistant (inbound). In certain instances, the CMM identifies the following message types:
text: Basic text message
card: A card representation that contains a title and, optionally, a description, image, and link.
attachment: A message with a media URL (file, image, video, or audio)
location: A message with geo-location coordinates
postback: A message with a postback payload
   Messages that are defined in CMM are channel-agnostic and can be created using CMM syntax. The channel-specific connectors transform the CMM message into the format required by the specific channel, allowing a user to run the digital assistant on multiple channels without the need to create separate message formats for each channel.

Lastly, the output pipeline 370 transmits the responses 372 to the end user such as via a user device or interface. In some instances, the responses 372 are rendered within a dialogue box of a GUI allowing the user to view and reply using the dialogue box (or alternative means such as a microphone). In other instances, the responses 372 are rendered within a dialogue box of a GUI having one or more GUI elements allowing for an easier response by the user. In this particular instance, a first response 372 (What is my current 401k Contribution?Also, can you tell me the contribution limit?) to the additional query 338 is rendered within the dialogue box of a GUI. Additionally, in order to follow-up on obtaining information still required for the initial utterance 302, the LLM 336 generates another response 372 prompting the user for the missing information (Would you like to change your contribution by percentage or amount?[Percentage][Amount]).

While the embodiment of computing environment 300 in FIG. 3 illustrates the digital assistant interacting in a particular conversation flow, this is not intended to be limiting and is merely provided to facilitate a better understanding of the role and responsibility of the components, services, models, and the like of the computing environment 300 within the conversation flow.

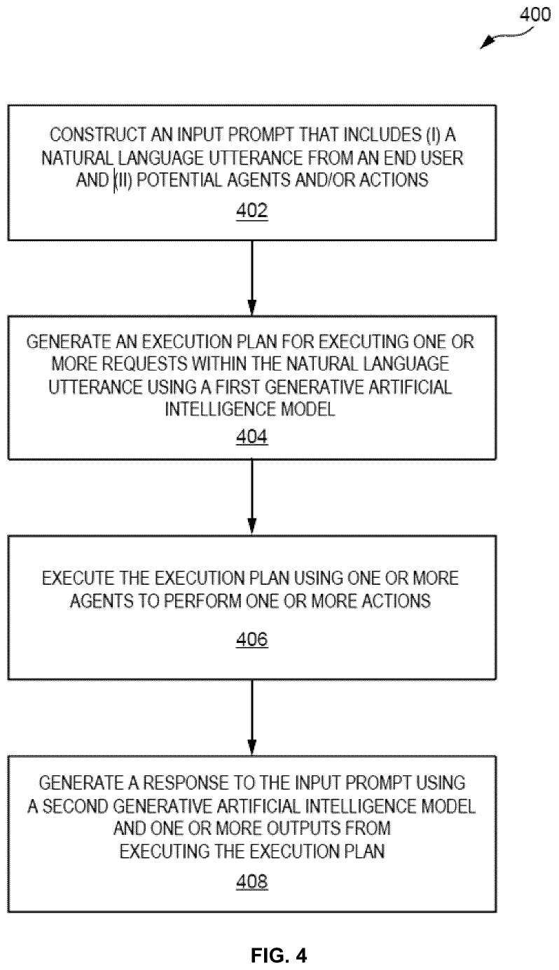

FIG. 4 is a flowchart of a process 400 for generating a response to user input using a digital assistant that can be implemented using generative artificial intelligence in accordance with various embodiments. The processing depicted in FIG. 4 may be implemented in software (e.g., code, instructions, program) executed by one or more processing units (e.g., processors, cores) of the respective systems, hardware, or combinations thereof. The software may be stored on a non-transitory storage medium (e.g., on a memory device). The process presented in FIG. 4 and described below is intended to be illustrative and non-limiting. Although FIG. 4 illustrates the various processing steps occurring in a particular sequence or order, this is not intended to be limiting. In certain alternative embodiments, the steps may be performed in some different order, or some steps may also be performed in parallel. In certain embodiments, the processing depicted in FIG. 4 may be performed by one or more of the components, computing devices, services, or the like, such as the digital assistant, the first and/or second generative artificial intelligence model (LLMs), etc., illustrated and described with respect to FIGS. 1-3.

At 402, an input prompt is constructed based on a natural language utterance received from a user of a digital assistant. The input prompt may be constructed by a digital assistant for input into a first generative artificial intelligence model (e.g., LLM).

In some instances, the input prompt is constructed based on the natural language utterance received from a user of the digital assistant and one or more candidate agents and associated actions identified from a data store of available agents and actions.

In some instances, constructing the input prompt comprises executing, using the natural language utterance, a sematic search on descriptions associated with the available agents and actions in the data store, identifying, based on the semantic search, the one or more potential agents and associated actions, and constructing a natural language representation for the input prompt by appending one or more potential agents and associated actions to the natural language utterance.

In some instances, the natural language utterance is a continuation or subsequent utterance within a conversation, and the input prompt further comprises: conversation history and actions executed prior to the natural language utterance. In some instances, constructing the input prompt comprises accessing the conversation history and the actions executed prior to the natural language utterance, and constructing the natural language representation for the input prompt by appending the one or more potential agents, the associated actions, and the conversation history and the actions executed prior to the natural language utterance.

In some instances, the one or more agents are a plurality of agents, and the one or more actions are a plurality of actions. A first subset of the plurality of agents and the plurality of actions may be in a first state and a second subset of the plurality of agents and the plurality of actions may be in a second state. The first state is a ready for execution state and the second state is not ready for execution state where additional information is required prior to execution of one or more actions within the second subset of the plurality of agents and the plurality of actions.

At 404, an execution plan is generated for executing one or more requests represented by the natural language utterance. The execution plan is generated by a first generative artificial intelligence model using the input prompt from 402.

In some instances, generating the execution plan comprises determining, based on the one or more potential agents and associated actions, one or more agents and one or more actions associated with the one or more agents that can service the one or more requests, and generating a structured output for the execution plan by creating an ordered list that comprises one or more actions (and optionally the one or more agents associated with the actions) for executing the one or more requests.

At 406, the execution plan is executed to perform the one or more actions using one or more agents. In some instances, executing the execution plan comprises triggering performance of the one or more actions by the one or more agents, and receiving one or more outputs from performance of the one or more actions by the one or more agents.

In some instances, executing the execution plan further comprises accessing contextual information that is needed by at least one of the one or more agents for performing at least one of the one or more actions, and triggering the performance of the one or more actions comprises forwarding one or more requests for performance of the one or more actions to the one the one or more agents. A request of the one or more requests being forwarded for performance of the at least one of the one or more actions may include the contextual information.

At 408, a response to the natural language utterance is generated by a second generative artificial intelligence model using the one or more outputs. The second generative artificial intelligence model may be similar or identical to, or may be different than, the first generative artificial intelligence model.

In some instances, the one or more agents are a plurality of agents, the one or more actions are a plurality of actions, and the one or more requests are a plurality of requests, and generating the execution plan further comprises determining whether one or more dependencies exist between the plurality of actions, and when the one or more dependencies exist, the ordered list is created to comprise the plurality agents, the plurality of actions for executing the one or more requests, and an indication of the one or more dependencies, when the execution plan comprises the indication of the one or more dependencies, the performance of the one or more actions by the one or more agents is triggered via serial processing, when the execution plan does not comprise the indication of the one or more dependencies, the performance of the one or more actions by the one or more agents is triggered via parallel processing, and the response is an aggregate response comprising a plurality of responses to the plurality of requests within the natural language utterance.

In some instances, the natural language utterance is a continuation or subsequent utterance within a conversation, and the response to the natural language utterance is generated by the second generative artificial intelligence model using the one or more output, the natural language utterance, and a conversation history for the conversation.

In some instances, the response is communicated to the user.

As used herein, when an action is “based on” something, this means the action can be based at least in part on at least a part of the something. As used herein, the terms “similarly,” “substantially,” “approximately,” and “about” are defined as being largely but not necessarily wholly what is specified (and include wholly what is specified) as understood by one of ordinary skill in the art. In any disclosed embodiment, the term “similarly,” “substantially,” “approximately,” or “about” may be substituted with “within [a percentage] of” what is specified, where the percentage includes 0.1 percent, 1 percent, 5 percent, and 10 percent, etc.

### Clinical Artificial Intelligence Agent (CAA)

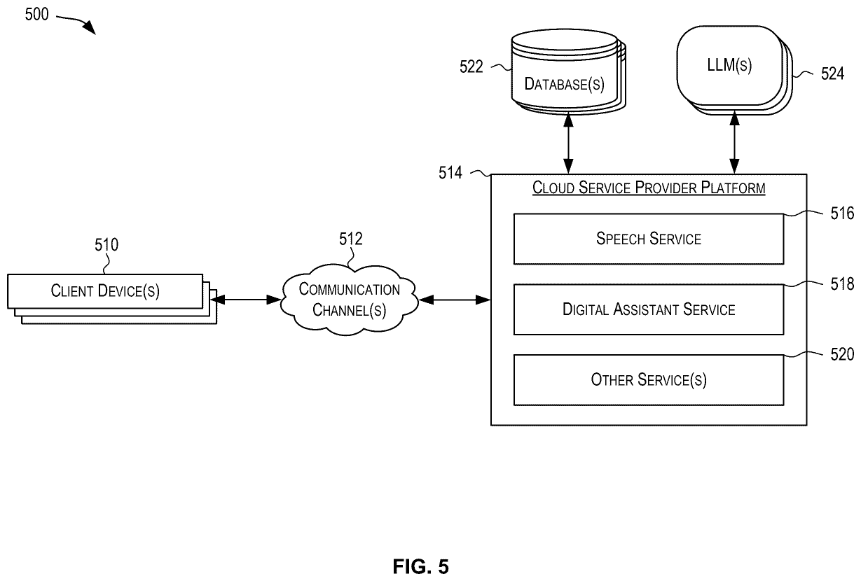

FIG. 5 shows a simplified diagram of an example environment 500 for a digital assistance service 518. As shown in FIG. 5, the environment 500 includes one or more client devices 510 (hereinafter “client devices 510”), one or more communication channels 512 (hereinafter “communication channels”), a cloud service provider platform 514 (hereinafter “platform 514”), one or more databases 522 (hereinafter “databases 522”), and one or more LLMs 524 (hereinafter “LLMs”). The platform 514, which can be included as part of a cloud infrastructure of a cloud service provider (e.g., Oracle Cloud Infrastructure or OCI), can be configured to communicate with, send data and information to, and receive data and information from the client devices 510 via the communication channels 512. Additionally, the platform 514 can be configured to access and/or call the databases 522 and the LLMs 524 to obtain and/or receive data and information from the databases 522 and the LLMs 524. Data and information received from the client devices 510, the databases 522, and the LLMs 524 can be used by the platform 514 to execute tasks and perform services such as a digital assistant service 518 that generates responses to a user query. While FIG. 5 shows the databases 522 and the LLMs 524 as being separate from the platform 514, this is not intended to be limiting, and one or more of the databases 522 and/or one or more of the LLMs 524 can be included as part of the platform 514 and/or the cloud infrastructure in which the platform 514 is included.

Each client device included in the client devices 510 can be any kind of electronic device that is capable of: executing applications; presenting information textually, graphically, and audibly such as via a display and a speaker; collecting information via one or more sensing elements such as image sensors, microphones, tactile sensors, touchscreen displays, and the like; connecting to a communication channel such as the communication channels 512 or a network such as a wireless network, wired network, a public network, a private network, and the like, to send and receive data and information; and/or storing data and information locally in one or more storage mediums of the electronic device and/or in one or more locations that are remote from the electronic device such as a cloud-based storage system, the platform 514, and/or the databases 522. Examples of electronic devices include, but are not limited to, mobile phones, desktop computers, portable computing devices, computers, workstations, laptop computers, tablet computers, and the like.

In some implementations, an application can be installed on, executing on, and/or accessed by a client device included in the client devices 510. The application and/or a user interface of the application can be utilized and/or interacted with (e.g., by an end user) to access, utilize, and/or interact with one or more services provided by the platform 514. The client device can be configured to receive multiple forms of input such as touch, text, voice, and the like, and the application can be configured to transform that input into one or more messages which can be transmitted or streamed to the platform 514 using one or more communication channels of the communication channels 512. Additionally, the client device can be configured to receive messages, data, and information from the platform 514 using one or more communication channels of the communication channels 512 and the application can be configured to present and/or render the received messages, data, and information in one or more user interfaces of the application.

Each communication channel included in the communication channels 512 can be any kind of communication channel that is capable of facilitating communication and the transfer of data and/or information between one or more entities such as the client devices 510, the platform 514, the databases 522, and the LLMs 524. Examples of communication channels include, but are not limited to, public networks, private networks, the Internet, wireless networks, wired networks, fiber optic networks, local area networks, wide area networks, and the like. The communication channels 512 can be configured to facilitate data and/or information streaming between and among the one or more entities. In some implementations, data and/or information can be streamed using one or more messages and according to one or more protocols. Each of the one or more messages can be a variable length message and each communication channel included in the communication channels 512 can include a stream orchestration layer that can receive the variable length message in accordance with a predefined interface, such as an interface defined using an interface description language like AsyncAPI. Each of the variable length messages can include context information that can be used to determine the route or routes for the variable length message as well as a text or binary payload of arbitrary length. Each of the routes can be configured using a polyglot stream orchestration language that is agnostic to the details of the underlying implementation of the routing tasks and destinations.

Each database included in the databases 522 can be any kind of database or knowledge base that is capable of storing data and/or information, providing access to data and/or information, and managing data and/or information. Data and/or information stored by each database can include data and/or information generated by, provided by, and/or otherwise obtained by the platform 514. Additionally, or alternatively, data and/or information stored and/or managed by each database can include data and/or information generated by, provided by, and/or otherwise obtained by other sources such as the client devices 510 and/or LLMs 524. One or more databases that are included in the databases 522 can be part of a platform for storing and managing healthcare information such as electronic health records for patients, electronic records of healthcare providers, and the like, and can store and manage electronic health records for patients of healthcare providers. An example platform is the Oracle Health Millenium Platform. Another example of a database that is included in the databases 522 is a semantic knowledge graph. Additionally, one or more databases included in the databases 522 can be provided by, managed by, and/or otherwise included as part of a cloud infrastructure of a cloud service provider (e.g., Oracle Cloud Infrastructure or OCI). Data and/or information stored and/or managed by the databases 522 can be accessed using one or more application programming interfaces (APIs) of the databases 522.

Each LLM included in the LLMs 524 can be any kind of LLM that is capable of obtaining or generating or retrieving one or more results in response to one or more inputs such as one or more prompts. Prompts for obtaining or generating or retrieving results from the LLMs 524 can obtained from or generated by or retrieved from or accessed from the client devices 510, the databases 512, the platform 514, the LLMs 524, and/or one or more other sources such as the Internet and other generative machine learning models. Each prompt can be configured to cause the LLMs 524 to perform one or more tasks, which causes one or more results to be provided or generated and the like. Prompts for the LLMs 524 can be pre-generated (i.e., before they are needed for a particular task) and/or generated in real-time (i.e., as they are needed for a particular task). In some implementations, prompts for the LLMs 524 can be engineered to achieve a desired result or results manually and/or by one or more machine-learning models. In some implementations, prompts for the LLMs 524 can be engineered one demand (i.e., in real-time and/or as needed) and/or at particular intervals (e.g., once per day, upon logging in by authenticated user into the platform 514). Each prompt of the one or more prompts can include a request for or a query for or a task to be performed by the LLMs 524 and contextual information. The contextual information can include information such as a text transcript or portions or segments thereof, information about an entity (e.g., information about a healthcare provider, information about a patient such as information included in an electronic health record for the patient, and the like), and/or other information or records (e.g., lab results, ambient temperature, and the like). LLMs included in the LLMs 524 can be pre-trained, fine-tuned, open source, off-the-shelf, licensed, subscribed to, and the like. Additionally, LLMs included in the LLMs 524 can include or have any size context window (i.e., can accept any number of tokens) and can be capable of interpreting complex instructions. One or more LLMs included in the LLMs 524 can be provided by, managed by, and/or otherwise included as part of the platform 514 and/or a cloud infrastructure of a cloud service provider (e.g., Oracle Cloud Infrastructure or OCI) that supports the platform 514. One or more LLMs included in the LLMs 524 can be accessed using one or more APIs of the LLMs 524 and/or a platform hosting or supporting or providing the LLMs 524.

As used herein, references to a “large language model” (LLM) is exemplary and non-limiting. The disclosed systems and methods are architecture-agnostic and apply to other model classes and sizes, including without limitation small language models (SLMs), large multimodel models (LMMs), multimodal large language models (MLLMs), vision-language models, speech-language models, encoder-only, decoder-only, and encoder-decoder transformers, convolutional and recurrent neural networks, graph neural networks, diffusion models, variational autoencoders (VAEs), generative adversarial networks (GANs), flow- or score-based models, retrieval-augmented models, ensembles, cascaded models, and hybrids thereof. Unless expressly stated otherwise, any functionality described with respect to an LMM may be implemented by any of the foregoing models and their equivalents.

The platform 514 can be configured to include various capabilities and provide various services to subscribers (e.g., end users) of the various services. In some implementations, in the case of an end user or subscriber being a healthcare provider, the healthcare provider can utilize the various services to facilitate the observation, care, treatment, management, and so on of their patient populations. For example, a healthcare provider can utilize the functionality provided by the various services provided by the platform 514 to examine and/or treat and/or facilitate the examination and/or treatment of a patient; view, edit, and/or manage a patient's electronic health record; perform administrative tasks such as scheduling appointments, managing patient populations, providing customer service to facilitate operation of a healthcare environment in which the healthcare provider practices, and so on.

In some implementations, the services provided by the platform 514 can include, but are not limited to, a speech service 516, the digital assistant service 518, and other service(s) 520 such as a SOAP Note service. The speech service 516 can be configured to convert audio into text such as a text transcript. For example, the speech service 516 can convert an audio recording of a conversation between a healthcare provider and a patient into a text transcript of the conversation. To convert audio into text, the speech service 516 can utilize one or more machine-learning models such as an automatic speech recognition (ASR) model. In the case that the audio is streamed to the platform 514 in the form of messages (as described above) with each message including a portion of the audio (e.g., a one second segment of the audio), in some implementations, the platform 514 and/or the speech service 516 can be configured to aggregate and combine all of the messages pertaining to the audio (e.g., all of the messages pertaining to a conversation) into audio data and/or an audio file prior to converting the audio data or audio file into text and/or a text transcript. In other implementations, the platform 514 and/or the speech service 516 can be configured to convert audio into text or a text transcript as the audio is received by the platform 514 and/or the speech service 516. The text or text transcript generated by the speech service 516 can be stored within the platform 514 and/or in another location such as in one or more databases of the databases 522, where it can be accessed by the platform 514, one or more other services of the platform 514 such as the digital assistant service 518 and/or SOAP note service 520, and/or the LLMs 524. Additionally, or alternatively, the text or text transcript generated by the speech service 516 can be provided to one or more other services of the platform 514 such as the digital assistant service 518 and/or the SOAP note service 520, and/or the LLMs 524.

The digital assistant service 518 can be configured to serve as an artificial intelligence-driven (AI-driven) conversational-type interface for the platform 514 that can conduct conversations with end users (e.g., those using the client devices 510) and perform functions and/or tasks based on the information conveyed by and/or ascertained from those conversations and other sources. The digital assistant service 518 can be configured with and/or configured to access natural language understanding (NLU) capabilities such as natural language processing, named entity recognition, intent classification, and so on. In some implementations, the digital assistant service 518 can be LLM-based and agent-driven in which agent(s) coordinate with LLM(s) for conducting conversations and performing functions and/or tasks such as the agentic digital assistant described above with respect to FIGS. 1-4.

The digital assistant service 518 can be configured to initiate a dialog, drive a previously initiated dialog (e.g., by responding to a turn in the dialog), and/or otherwise participate in a conversation. In some implementations, the digital assistant service 518 can drive and/or participate in a dialog and/or conversation in response to events that have occurred at the client devices 510, the platform 514, the databases 522, the LLMs 524, and/or at the cloud infrastructure supporting the platform 514. In the case of an LLM-based and agent-drive digital assistant service 518, events can be mapped to a particular prompt or prompts to retrieve a result or results for the prompt or prompts, which can then be used to render the user interface. In some implementations, the digital assistant service 518 can drive and/or participate in a dialog and/or conversation in response to messages received from the client devices 510, the platform 514, the databases 522, the LLMs 524, and/or at the cloud infrastructure supporting the platform 514. In the case of an LLM-based and agent-driven digital assistant service 18, the metadata included in the messages can be used to generate and/or access a particular prompt or prompts to retrieve a result and/or results that can be used to render the user interface.

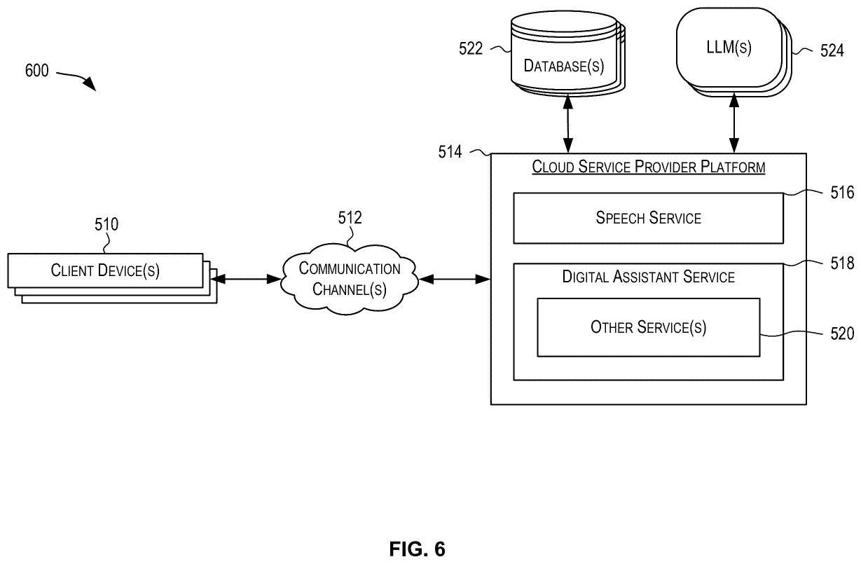

Although not shown, the platform 514 can include other capabilities and services such as authentication services, management services, task management services, notification services, and the like. The various capabilities and services of the platform 514 can be implemented utilizing one or more computing resources and/or servers of the platform 514 and provided by the platform 514 by way of subscriptions. Additionally, or alternatively, while FIG. 5 shows the services of the platform 514 as being separate services, one or more of the services can be combined with other services and/or be considered to be a sub-service of another service. For example, as shown in FIG. 6, in the environment 600, the other service(s) 520 can provide a sub-service of or part of the digital assistant service 518.

The environments 500 and 600 depicted in FIGS. 5 and 6 are merely exemplary and are not intended to unduly limit the scope of claimed embodiments. One of ordinary skill in the art would recognize many possible variations, alternatives, and modifications. For example, in some implementations, the environments 500 and 600 can be implemented using more or fewer services than those shown in FIG. 5 and FIG. 6, may combine two or more services, or may have a different configuration or arrangement of services.

### Medical Concept Expansion Agent

Clinicians often face challenges in retrieving relevant information quickly due to the complexity of Electronic Health Record (EHR) systems and the structured nature of the data. In some examples, a data agent provides a more efficient and intuitive access to structured patient data within Electronic Health Records (EHRs) compared to prior systems. Using techniques described herein, relevant information to a query can be located quickly and accurately.

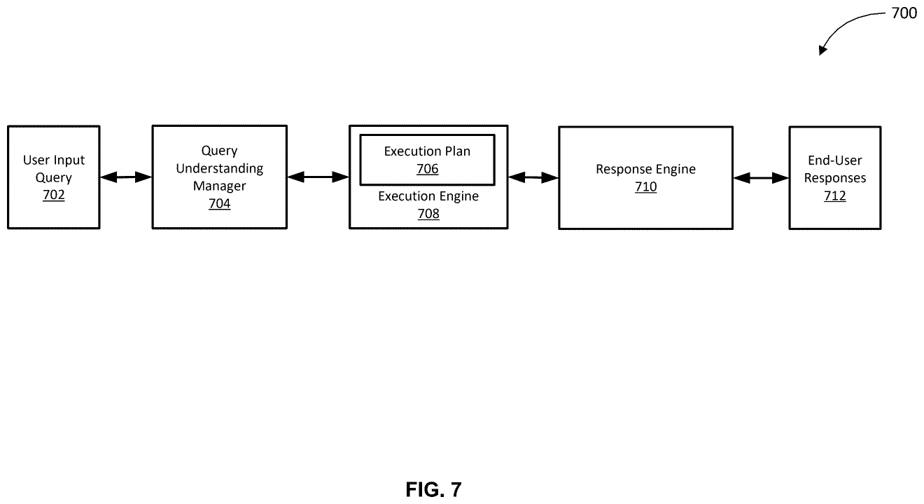

FIG. 7 is an example of an architecture for a computing environment 700 for a digital assistant implemented with generative artificial intelligence in accordance with various embodiments. The computing environment 700 can be configured the same as the computing environments 300, 500, 600. In FIG. 7, components of the computing environment 700 that can be configured the same as the corresponding components in the computing environment 300, 500, 600 are designated with the same reference identifier numbers and the description for these components set forth above with regard to the computing environment 300 is applicable to these components in the computing environment 700.

As illustrated in FIG. 7, an infrastructure and various services and features can be used to enable a user to interact with a digital assistant (e.g., digital assistant 115A described with respect to FIG. 1) based at least in part on a series of prompts such as a conversation. The following is a detailed walkthrough of a conversation flow and the role and responsibility of the components, services, models, and the like of the computing environment 700 within the conversation flow. In this walkthrough, it is assumed that a user (e.g., a “a health professional”) is interested in asking a question about a patient. As illustrated, FIG. 7 includes a user input query 702 (e.g., a query), a query understanding manager 704, an execution engine 708, a response engine 710, and end-user responses 712.

Referring to FIG. 7, an utterance 702 (e.g., “Hi, has she gotten any imaging done?”, “Has she had a urine analysis yet?”, “What sensitivities does she have in her cultures?”, . . . ) can be communicated to the digital assistant (e.g., via text dialogue box or microphone) and provided as input to the query understanding manager 704. According to some configurations, the query understanding manager 704 uses NLP and optionally machine learning techniques to understand the meaning of the user query 702. In some cases, a semantic search is performed by the query understanding manager 704 that takes into account the relationships between words, the context of the query, synonyms, and other linguistic nuances. This allows the digital assistant to provide more accurate and contextually relevant results, making it more effective in understanding the user's intent in the utterance 702.

In some cases, the query understanding manager 704 identifies the medical concepts that are included within the query. According to some examples, the query understanding manager 704 identifies the key phrases/entities associated with one or more medical concepts within the query 702. According to some configurations, the medical concepts can be represented by a medical coding system such as one or more of Systematized Nomenclature of Medicine—Clinical Terms (SNOMED CT), Prescription Normalization (RxNORM), Logical Observation Identifiers Names and Codes (LOINC), International Classification of Diseases (ICD-10), and the like.

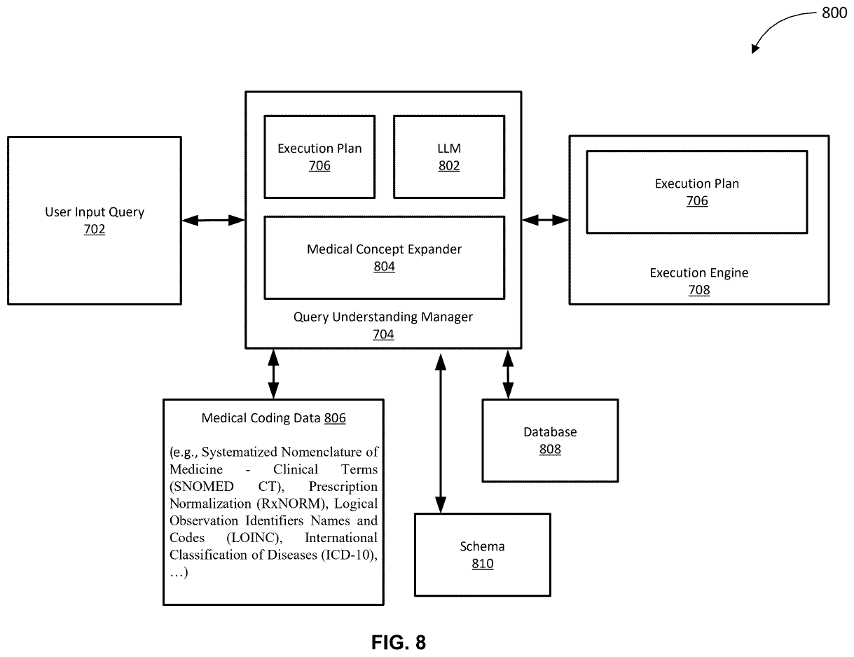

In some examples, the identified medical concepts are expanded by the query understanding manager 704 to include one or more other medical concepts that are related to the identified medical concepts (See FIG. 8 and related discussion for further details).

In some examples, a Fast Healthcare Interoperability Resources (FHIR)-compliance data structure can be generated that helps ensure consistency in the recording and reporting medical information and Interoperability among platforms. FHIR may refer to a standard for exchanging healthcare information electronically between different systems, which may utilize modern web technologies such as RESTful APIs and JSON/XML format to facilitate the exchange. By identifying the related medical concepts, the scope of the search can be broadened without also having to search unrelated medical concepts.

After identifying the medical concepts within the query and other medical concepts that are similar, the query understanding manager 704 identifies the portions of a schema associated with the medical concepts associated with one or more medical coding systems. Generally, the portions of the schema associated with the identified medical condition are obtained by the query understanding manager 704. In some configurations, the query understanding manager 704 combines the portions of a database schema that represents the identified medical concepts within the query and the portions of the database schema that represents the medical concepts identified to be similar.

In some examples, a prompt (that can be provided to a machine learning model) is generated by the query understanding manager 704 that comprises an instruction, the portions of the database schema associated with the identified medical concepts, and at least a portion of the received user input received (e.g., an utterance associated with a natural language component of the query). In some configurations, generating the prompt can include providing the one or more portions of the database schema to a second machine learning model to obtain structured object query language that can be used to obtain information from one or more databases. The generated prompt can then be provided to an execution engine 708 to execute the associated execution plan 706 that generates a response to the query. In some examples, the generated prompt includes SOQL query language generated by a data action planner LLM used by the query understanding manager.

As described above, an execution plan 706 can be generated based on the use input query 702. In some examples, the LLM 802 has a deep generative model architecture (e.g., a reversible or autoregressive architecture with) for generating the execution plan 706. In some instances, the LLM 802 has over 100 billion parameters and generates the execution plan 706 using autoregressive language modeling within a transformer architecture, allowing the LLM 802 to capture complex patterns and dependencies in the user input query 702. The LLM's 802 ability to generate the execution plan 706 is a result of its training on diverse and extensive textual data, enabling the LLM to understand human language across a wide range of contexts. During training, the LLM 802 learns to predict the next word in a sequence given the context of the preceding words. This process involves adjusting the model's parameters (weights and biases) based on the errors between its predictions and the actual next words in the training data. When the LLM 802 receives an input such as the input query 702, the LLM 802 tokenizes the text into smaller units such as words or sub-words. Each token is then represented as a vector in a high-dimensional space. The LLM 802 processes the input sequence token by token, maintaining an internal representation of context. The LLM's 802 attention mechanism allows it to weigh the importance of different tokens in the context of generating the next word. For each token in the vocabulary, the LLM 802 calculates a probability distribution based on its learned parameters. This probability distribution represents the likelihood of each token being the next word given the context. To generate the execution plan 706, the LLM 802 samples a token from the calculated probability distribution. The sampled token becomes the next word in the generated sequence. This process is repeated iteratively, with each newly generated token influencing the context for generating the subsequent token. The LLM 802 can continue generating tokens until a predefined length or stopping condition is reached.

After identifying the medical concepts within the query and other medical concepts that are similar, one or more portions of a schema associated with the medical concepts are identified (e.g., from one or more medical coding systems) by the query understanding manager 704.

Generally, the portions of the schema associated with the identified medical condition are obtained from a data store. In some configurations, the query understanding manager 704 combines the portions of a database schema that represents the identified medical concepts within the query and the portions of the database schema that represents the medical concepts identified to be similar.

In some examples, a prompt (that may be provided to a machine learning model) is generated by the query understanding manager 704 that comprises an instruction, the portions of the database schema associated with the identified medical concepts, and at least a portion of the received user input received (e.g., an utterance associated with a natural language component of the query). In some configurations, generating the prompt can include providing the one or more portions of the database schema to a second machine learning model to obtain structured object query language that can be used to obtain information from one or more databases (e.g., associated with EHRs). The generated prompt can then be provided to an execution engine 708 to execute the associated execution plan 706 that generates a response to the query. In some examples, the generated prompt includes SOQL query language generated by a data action planner LLM used by the query understanding manager. The query result can then be provided to the user who made the query.

The execution plan 706 includes an ordered list of agents and/or actions that can be used and/or executed to sufficiently respond to the request. The execution plan 706 is then executed by execution engine 708. The execution engine 708 may include a number of engines, such as but not limited to a natural language-to-programming language translator, a knowledge engine, an API engine, a prompt engine, and the like, for executing the actions of agents and implementing the execution plan 708. For example, the natural language-to-programming language translator, such as a Conversation to Oracle Meaning Representation Language (C2OMRL) model, may be used by an agent to translate natural language into a intermedial logical for (e.g., OMRL), convert the intermediate logical form into a system programming language (e.g., SQL, SOQL, . . . ) and execute the system programming language (e.g., execute an SQL query, SOQL query, . . . ) on an a data store to execute actions and/or obtain data or information. A prompt engine may be used by an agent to construct a prompt for input into an LLM such as an LLM.

In some instances, knowledge is retrieved by the execution engine 708 from one or more assets (e.g., files/documents, memory, . . . ). Knowledge retrieval and action execution may be implemented using various techniques including internal task mapping and/or machine learning models such as additional LLM models. For example, the query and associated agent for “has she gotten any imaging done?” may be mapped to a ‘semantic search’ knowledge task type for searching one or more indices for a response to a given query. By way of another example, a request such as “Can you summarize the imaging performed” can be or include a ‘summary’ knowledge task type that may be mapped to a different index. Over time, a library of generic end-user task or action types (e.g., semantic search, summarization, compare/contrast, heterogeneous data synthesis, etc.) may be built to ensure that the indices and models are optimized to the various task or action types.

The result of implementing the execution plan 706 is output data (e.g., results of actions, data, information, etc.), which is transmitted to response engine 710 for generating end-user responses 712. An LLM (as discussed herein) generates end-user responses 712. In some instances, the LLM uses a deep generative model architecture (e.g., a reversible or autoregressive architecture with) for generating the responses 712.

In some instances, the response engine 710 generates end-user responses 712 may be in the format of a Conversation Message Model (CMM) and output as rich multi-modal responses.

The CMM defines the various message types that the digital assistant can send to the user (outbound), and the user can send to the digital assistant (inbound). In certain instances, the CMM identifies the following message types:
text: Basic text message
card: A card representation that contains a title and, optionally, a description, image, and link.
attachment: A message with a media URL (file, image, video, or audio)
location: A message with geo-location coordinates
postback: A message with a postback payload
   Messages that are defined in CMM are channel-agnostic and can be created using CMM syntax. The channel-specific connectors transform the CMM message into the format required by the specific channel, allowing a user to run the digital assistant on multiple channels without the need to create separate message formats for each channel.

Lastly, the response engine 710 transmits the responses 712 to the end user such as via a user device or interface. In some instances, the responses 712 are rendered within a dialogue box of a GUI allowing the user to view and reply using the dialogue box (or alternative means such as a microphone). In other instances, the responses 712 are rendered within a dialogue box of a GUI having one or more GUI elements allowing for an easier response by the user.

While the embodiment of computing environment 700 in FIG. 7 illustrates the digital assistant interacting in a particular conversation flow, this is not intended to be limiting and is merely provided to facilitate a better understanding of the role and responsibility of the components, services, models, and the like of the computing environment 700 within the conversation flow.

FIG. 8 is an example of an architecture for a computing environment 800 for a digital assistant implemented with generative artificial intelligence in accordance with various embodiments. The computing environment 800 can be configured the same as the computing environments 300, 500, 600, 700. FIG. 8 is similar to FIG. 7 but includes more details. In FIG. 8, components of the computing environment 800 that can be configured the same as the corresponding components in the computing environment 300, 500, 600, 700 are designated with the same reference identifier numbers and the description for these components set forth above with regard to the computing environment 300 is applicable to these components in the computing environment 800.

As illustrated in FIG. 8, an infrastructure and various services and features can be used to enable a user to interact with a digital assistant (e.g., digital assistant 115A described with respect to FIG. 1) based at least in part on a series of prompts such as a conversation. The following is a detailed walkthrough of a conversation flow and the role and responsibility of the components, services, models, and the like of the computing environment 800 within the conversation flow. In this walkthrough, it is assumed that a user (e.g., a “a health professional”) is interested in asking a question about a patient. As illustrated, FIG. 8 includes a user input query 702 (e.g., a query), a query understanding manager 704, an execution engine 708, a response engine 710, and end-user responses 712. The query understanding manager 704 includes an LLM 802, and a medical concept expander 804. The query understanding manager 704 is connected to medical coding data 806 and database 808.

Referring to FIG. 8, the query understanding manager 704 identifies the medical concepts that are included within the query. According to some examples, the query understanding manager 704 identifies the key phrases/entities associated with one or more medical concepts within the query 702. In some examples, the query understanding manager 704 can access medical coding data 806 that includes data from one or more medical coding systems such as one or more of Systematized Nomenclature of Medicine—Clinical Terms (SNOMED CT), Prescription Normalization (RxNORM), Logical Observation Identifiers Names and Codes (LOINC), International Classification of Diseases (ICD-10), and the like.

In some examples, the query understanding manager 704 uses a medical concept expander component 804 to identify medical concepts that are similar to the medical concepts identified in the user input query 702. According to some configurations, the query understanding manager 704 performs a search of a vector database, such as database 808, that includes embeddings of medical concepts (including medical codes and other relevant information) of one or more medical coding systems (e.g., SNOMED-CT, RxNORM, . . . ) is performed to identify medical concepts that are similar to the medical concepts identified in the query.

According to some configurations, the similarity search performed by the query understanding manager 704 uses an embeddings-based Approximate Nearest Neighbor (ANN) search algorithm, which is a search approach that can use various similarity metrics, including cosine similarity (as a distance metric). Those skilled in the art will appreciate various ways and techniques that can be used to perform similarity searches. The techniques may include, but are not limited to, ANN search algorithm, Locality-sensitive hashing (LSH), K-nearest neighbors (KNN), and the like.

In some examples, the search returns a ranked list of medical concepts based on the identified medical concepts and their associated similarity scores. At least a portion of the medical concepts are selected by the medical concept expander 804 from the ranked list according to certain criteria (e.g., top ten of all medical concepts in the list, top two from each medical entity type in the ranked list, within a specified range of a similarity score, . . . ) to expand the medical concepts..

After identifying the medical concepts within the query and other medical concepts that are similar, the query understanding manager 704 identifies the portions of a schema from schema 810 that associated with the medical concepts. Generally, the portions of the schema associated with the identified medical are obtained from the schema data store 810 by the query understanding manager 704. In some configurations, the query understanding manager 704 combines the portions of a database schema that represents the identified medical concepts within the query and the portions of the database schema that represents the medical concepts identified to be similar.

While the embodiment of computing environment 800 in FIG. 8 illustrates the digital assistant interacting in a particular conversation flow, this is not intended to be limiting and is merely provided to facilitate a better understanding of the role and responsibility of the components, services, models, and the like of the computing environment 800 within the conversation flow.

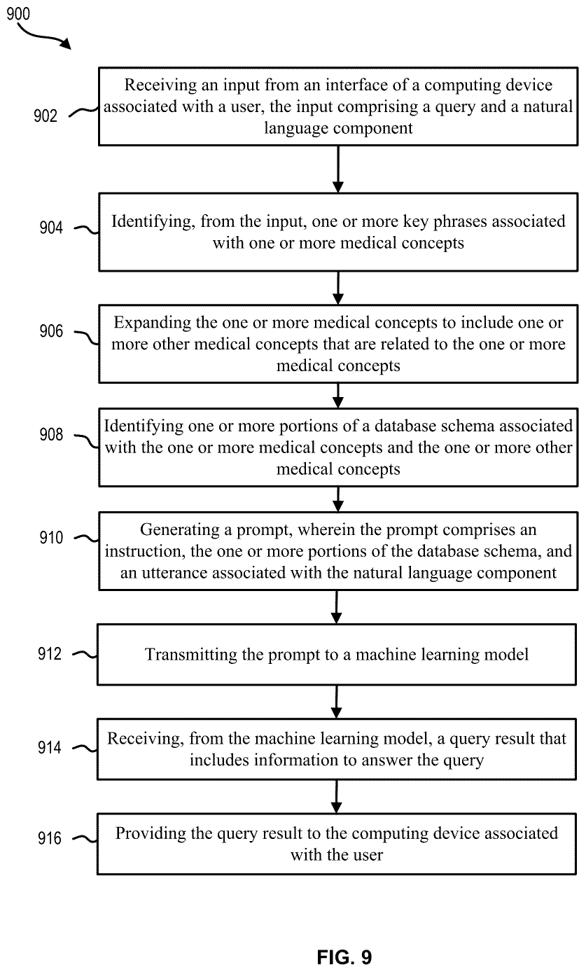

FIG. 9 is a simplified block diagram a method 900 of generating a response to a user query, in accordance with various embodiments. Any suitable computing environment, such as the computing environment 700 described herein, can be used to practice the method 900.

At step 902, an input from an interface of a computing device associated with a user is received. In some examples, the input comprises a query and a natural language component. In some examples described herein, the query can be a physician inquiry, or some other authorized health professional query, related to a patient.

At step 904, one or more key phrases associated with one or more medical concepts are identified from the input. In some examples, the query understanding manager 704 identifies the key phrases/entities within the input that are associated with any medical concepts. In some configurations, the query understanding manager 704 may use one or more machine learning models to identify the medical concepts within the input. According to some configurations, the medical concepts can be represented by a medical coding system such as one or more of Systematized Nomenclature of Medicine—Clinical Terms (SNOMED CT), Prescription Normalization (RxNORM), Logical Observation Identifiers Names and Codes (LOINC), International Classification of Diseases (ICD-10), and the like. In some examples, the medical concepts can be organized into hierarchies (e.g., from general to more specific).

At step 906, the identified medical concepts are expanded to include one or more other medical concepts that are related to the identified medical concept(s). In some examples, expanding the one or more medical concepts comprises determining similar medical concepts identified within a medical coding system. As discussed above, determining the similar medical concepts can include performing a search of a vector database that contains embeddings of medical concepts of the medical coding system. In other examples, related medical concepts can be determined based on the relationships of the identified medical concepts defined within one or more of the medical coding systems. In yet other examples, similar medical concepts can be determined by providing the identified medical concepts to an LLM and receiving possible medical concepts that are similar from the LLM.

At step 908, one or more portions of a schema associated with the one or more medical concepts and the one or more other medical concepts are identified. As discussed above, the query understanding manager 704 identifies the portions of the schema associated with the identified medical concepts and the other medical concepts that were identified to be similar. In some configurations, the query understanding manager 704 combines the portions of a database schema that represents the identified medical concepts within the query and the portions of the database schema that represents the medical concepts identified to be similar.

At step 910, a prompt is generated. In some example, the prompt comprises an instruction, the portions of the database schema associated with the identified medical concepts, and at least a portion of the user input received at 902, such as an utterance associated with the natural language component. In some configurations, generating the prompt can include providing the one or more portions of the database schema to a second machine learning model to obtain structured object query language that can be used to obtain information from one or more databases.

At step 912, the generated prompt is transmitted to a machine learning model. As discussed above, the generated prompt can be provided to the execution engine 708 to execute the associated execution plan 706. In some examples, the generated prompt includes SOQL query language generated by a data action planner LLM used by the query understanding manager 704.

At step 914, a query result that includes information to answer the query is received from the machine learning model. As discussed above, the query result is an answer the user input query 702.

At step 916, providing the query result to the computing device associated with the user.

### Examples of Cloud Infrastructure Architectures

The term cloud service is generally used to refer to a service that is made available by a cloud service provider (CSP) to users (e.g., cloud service customers) on demand (e.g., via a subscription model) using systems and infrastructure (cloud infrastructure) provided by the CSP. Typically, the servers and systems that make up the CSP's infrastructure are separate from the user's own on-premise servers and systems. Users can thus avail themselves of cloud services provided by the CSP without having to purchase separate hardware and software resources for the services. Cloud services are designed to provide a subscribing user easy, scalable access to applications and computing resources without the user having to invest in procuring the infrastructure that is used for providing the services.

There are several cloud service providers that offer various types of cloud services. As discussed herein, there are various types or models of cloud services including IaaS, software as a service (SaaS), platform as a service (PaaS), and others. A user can subscribe to one or more cloud services provided by a CSP. The user can be any entity such as an individual, an organization, an enterprise, and the like. When a user subscribes to or registers for a service provided by a CSP, a tenancy or an account is created for that user. The user can then, via this account, access the subscribed-to one or more cloud resources associated with the account.

As noted above, IaaS is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance.

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, provides a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand) or the like.

In some examples, IaaS provisioning may refer to acquiring computers or virtual hosts for use and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

FIG. 10 is a block diagram 1000 illustrating an example pattern of an IaaS architecture, according to at least one embodiment. Service operators 1002 can be communicatively coupled to a secure host tenancy 1004 that can include a virtual cloud network (VCN) 1006 and a secure host subnet 1008. In some examples, the service operators 1002 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 6, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially available UNIX® or UNIX-like operating systems, including without limitation the variety of GNU/Linux operating systems, such as, for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 1006 and/or the Internet.

The VCN 1006 can include a local peering gateway (LPG) 1010 that can be communicatively coupled to a secure shell (SSH) VCN 1012 via an LPG 1010 contained in the SSH VCN 1012. The SSH VCN 1012 can include an SSH subnet 1014, and the SSH VCN 1012 can be communicatively coupled to a control plane VCN 1016 via the LPG 1010 contained in the control plane VCN 1016. Also, the SSH VCN 1012 can be communicatively coupled to a data plane VCN 1018 via an LPG 1010. The control plane VCN 1016 and the data plane VCN 1018 can be contained in a service tenancy 1019 that can be owned and/or operated by the IaaS provider.

The control plane VCN 1016 can include a control plane demilitarized zone (DMZ) tier 1020 that acts as a perimeter network (e.g., portions of a corporate network between the corporate intranet and external networks). The DMZ-based servers may have restricted responsibilities and help keep breaches contained. Additionally, the control plane DMZ tier 1020 can include one or more load balancer (LB) subnet(s) 1022, a control plane app tier 1024 that can include app subnet(s) 1026, a control plane data tier 1028 that can include database (DB) subnet(s) 1030 (e.g., frontend DB subnet(s) and/or backend DB subnet(s)). The LB subnet(s) 1022 contained in the control plane DMZ tier 1020 can be communicatively coupled to the app subnet(s) 1026 contained in the control plane app tier 1024 and an Internet gateway 1034 that can be contained in the control plane VCN 1016, and the app subnet(s) 1026 can be communicatively coupled to the DB subnet(s) 1030 contained in the control plane data tier 1028 and a service gateway 1036 and a network address translation (NAT) gateway 1038. The control plane VCN 1016 can include the service gateway 1036 and the NAT gateway 1038.

The control plane VCN 1016 can include a data plane mirror app tier 1040 that can include app subnet(s) 1026. The app subnet(s) 1026 contained in the data plane mirror app tier 1040 can include a virtual network interface controller (VNIC) 1042 that can execute a compute instance 1044. The compute instance 1044 can communicatively couple the app subnet(s) 1026 of the data plane mirror app tier 1040 to app subnet(s) 1026 that can be contained in a data plane app tier 1046.

The data plane VCN 1018 can include the data plane app tier 1046, a data plane DMZ tier 1048, and a data plane data tier 1060. The data plane DMZ tier 1048 can include LB subnet(s) 1022 that can be communicatively coupled to the app subnet(s) 1026 of the data plane app tier 1046 and the Internet gateway 1034 of the data plane VCN 1018. The app subnet(s) 1026 can be communicatively coupled to the service gateway 1036 of the data plane VCN 1018 and the NAT gateway 1038 of the data plane VCN 1018. The data plane data tier 1060 can also include the DB subnet(s) 1030 that can be communicatively coupled to the app subnet(s) 1026 of the data plane app tier 1046.

The Internet gateway 1034 of the control plane VCN 1016 and of the data plane VCN 1018 can be communicatively coupled to a metadata management service 1062 that can be communicatively coupled to public Internet 1064. Public Internet 1064 can be communicatively coupled to the NAT gateway 1038 of the control plane VCN 1016 and of the data plane VCN 1018. The service gateway 1036 of the control plane VCN 1016 and of the data plane VCN 1018 can be communicatively coupled to cloud services 1067.

In some examples, the service gateway 1036 of the control plane VCN 1016 or of the data plane VCN 1018 can make application programming interface (API) calls to cloud services 1067 without going through public Internet 1064. The API calls to cloud services 1067 from the service gateway 1036 can be one-way: the service gateway 1036 can make API calls to cloud services 1067, and cloud services 1067 can send requested data to the service gateway 1036. But, cloud services 1067 may not initiate API calls to the service gateway 1036.

In some examples, the secure host tenancy 1004 can be directly connected to the service tenancy 1019, which may be otherwise isolated. The secure host subnet 1008 can communicate with the SSH subnet 1014 through an LPG 1010 that may enable two-way communication over an otherwise isolated system. Connecting the secure host subnet 1008 to the SSH subnet 1014 may give the secure host subnet 1008 access to other entities within the service tenancy 1019.

The control plane VCN 1016 may allow users of the service tenancy 1019 to set up or otherwise provision desired resources. Desired resources provisioned in the control plane VCN 1016 may be deployed or otherwise used in the data plane VCN 1018. In some examples, the control plane VCN 1016 can be isolated from the data plane VCN 1018, and the data plane mirror app tier 1040 of the control plane VCN 1016 can communicate with the data plane app tier 1046 of the data plane VCN 1018 via VNICs 1042 that can be contained in the data plane mirror app tier 1040 and the data plane app tier 1046.

In some examples, users of the system, or customers, can make requests, for example create, read, update, or delete (CRUD) operations, through public Internet 1064 that can communicate the requests to the metadata management service 1062. The metadata management service 1062 can communicate the request to the control plane VCN 1016 through the Internet gateway 1034. The request can be received by the LB subnet(s) 1022 contained in the control plane DMZ tier 1020. The LB subnet(s) 1022 may determine that the request is valid, and in response to this determination, the LB subnet(s) 1022 can transmit the request to app subnet(s) 1026 contained in the control plane app tier 1024. If the request is validated and requires a call to public Internet 1064, the call to public Internet 1064 may be transmitted to the NAT gateway 1038 that can make the call to public Internet 1064. Metadata that may be desired to be stored by the request can be stored in the DB subnet(s) 1030.

In some examples, the data plane mirror app tier 1040 can facilitate direct communication between the control plane VCN 1016 and the data plane VCN 1018. For example, changes, updates, or other suitable modifications to configuration may be desired to be applied to the resources contained in the data plane VCN 1018. Via a VNIC 1042, the control plane VCN 1016 can directly communicate with, and can thereby execute the changes, updates, or other suitable modifications to configuration to, resources contained in the data plane VCN 1018.

In some embodiments, the control plane VCN 1016 and the data plane VCN 1018 can be contained in the service tenancy 1019. In this case, the user, or the customer, of the system may not own or operate either the control plane VCN 1016 or the data plane VCN 1018. Instead, the IaaS provider may own or operate the control plane VCN 1016 and the data plane VCN 1018, both of which may be contained in the service tenancy 1019. This embodiment can enable isolation of networks that may prevent users or customers from interacting with other users', or other customers', resources. Also, this embodiment may allow users or customers of the system to store databases privately without needing to rely on public Internet 1064, which may not have a desired level of threat prevention, for storage.

In other embodiments, the LB subnet(s) 1022 contained in the control plane VCN 1016 can be configured to receive a signal from the service gateway 1036. In this embodiment, the control plane VCN 1016 and the data plane VCN 1018 may be configured to be called by a customer of the IaaS provider without calling public Internet 1064. Customers of the IaaS provider may desire this embodiment since database(s) that the customers use may be controlled by the IaaS provider and may be stored on the service tenancy 1019, which may be isolated from public Internet 1064.

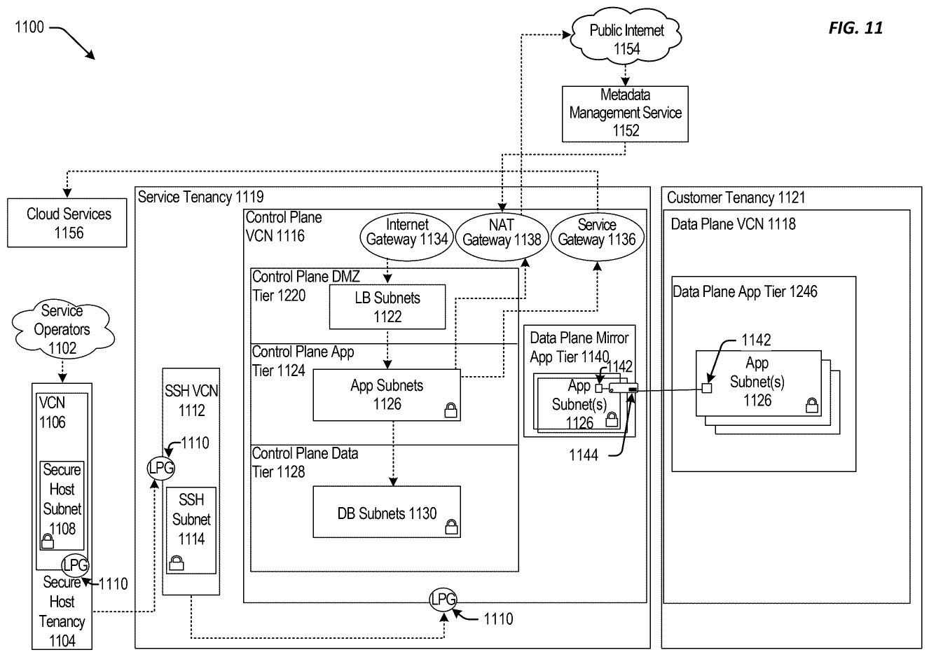

FIG. 11 is a block diagram 1100 illustrating another example pattern of an IaaS architecture, according to at least one embodiment. Service operators 1102 (e.g., service operators 1002 of FIG. 10) can be communicatively coupled to a secure host tenancy 1104 (e.g., the secure host tenancy 1004 of FIG. 10) that can include a virtual cloud network (VCN) 1106 (e.g., the VCN 1006 of FIG. 10) and a secure host subnet 1108 (e.g., the secure host subnet 1008 of FIG. 10). The VCN 1006 can include a local peering gateway (LPG) 1110 (e.g., the LPG 1010 of FIG. 10) that can be communicatively coupled to a secure shell (SSH) VCN 1112 (e.g., the SSH VCN 1012 of FIG. 10) via an LPG 1110 contained in the SSH VCN 1112. The SSH VCN 1112 can include an SSH subnet 1114 (e.g., the SSH subnet 1014 of FIG. 10), and the SSH VCN 1112 can be communicatively coupled to a control plane VCN 1116 (e.g., the control plane VCN 1016 of FIG. 10) via an LPG 1110 contained in the control plane VCN 1116. The control plane VCN 1116 can be contained in a service tenancy 1119 (e.g., the service tenancy 1019 of FIG. 10), and the data plane VCN 1118 (e.g., the data plane VCN 1018 of FIG. 10) can be contained in a customer tenancy 1121 that may be owned or operated by users, or customers, of the system.

The control plane VCN 1116 can include a control plane DMZ tier 1120 (e.g., the control plane DMZ tier 1020 of FIG. 10) that can include LB subnet(s) 1122 (e.g., LB subnet(s) 1022 of FIG. 10), a control plane app tier 1124 (e.g., the control plane app tier 1024 of FIG. 10) that can include app subnet(s) 1126 (e.g., app subnet(s) 1026 of FIG. 10), a control plane data tier 1128 (e.g., the control plane data tier 1028 of FIG. 10) that can include database (DB) subnet(s) 1130 (e.g., similar to DB subnet(s) 1030 of FIG. 10). The LB subnet(s) 1122 contained in the control plane DMZ tier 1120 can be communicatively coupled to the app subnet(s) 1126 contained in the control plane app tier 1124 and an Internet gateway 1134 (e.g., the Internet gateway 1034 of FIG. 10) that can be contained in the control plane VCN 1116, and the app subnet(s) 1126 can be communicatively coupled to the DB subnet(s) 1130 contained in the control plane data tier 1128 and a service gateway 1136 (e.g., the service gateway 1036 of FIG. 10) and a network address translation (NAT) gateway 1138 (e.g., the NAT gateway 1038 of FIG. 10). The control plane VCN 1116 can include the service gateway 1136 and the NAT gateway 1138.

The control plane VCN 1116 can include a data plane mirror app tier 1140 (e.g., the data plane mirror app tier 1040 of FIG. 10) that can include app subnet(s) 1126. The app subnet(s) 1126 contained in the data plane mirror app tier 1140 can include a virtual network interface controller (VNIC) 1142 (e.g., the VNIC of 1042) that can execute a compute instance 1144 (e.g., similar to the compute instance 1044 of FIG. 10). The compute instance 1144 can facilitate communication between the app subnet(s) 1126 of the data plane mirror app tier 1140 and the app subnet(s) 1126 that can be contained in a data plane app tier 1146 (e.g., the data plane app tier 1046 of FIG. 10) via the VNIC 1142 contained in the data plane mirror app tier 1140 and the VNIC 1142 contained in the data plane app tier 1146.

The Internet gateway 1134 contained in the control plane VCN 1116 can be communicatively coupled to a metadata management service 1152 (e.g., the metadata management service 1062 of FIG. 10) that can be communicatively coupled to public Internet 1154 (e.g., public Internet 1064 of FIG. 10). Public Internet 1154 can be communicatively coupled to the NAT gateway 1138 contained in the control plane VCN 1116. The service gateway 1136 contained in the control plane VCN 1116 can be communicatively coupled to cloud services 1156 (e.g., cloud services 1067 of FIG. 10).

In some examples, the data plane VCN 1118 can be contained in the customer tenancy 1121. In this case, the IaaS provider may provide the control plane VCN 1116 for each customer, and the IaaS provider may, for each customer, set up a unique compute instance 1144 that is contained in the service tenancy 1119. Each compute instance 1144 may allow communication between the control plane VCN 1116, contained in the service tenancy 1119, and the data plane VCN 1118 that is contained in the customer tenancy 1121. The compute instance 1144 may allow resources, which are provisioned in the control plane VCN 1116 that is contained in the service tenancy 1119, to be deployed or otherwise used in the data plane VCN 1118 that is contained in the customer tenancy 1121.

In other examples, the customer of the IaaS provider may have databases that live in the customer tenancy 1121. In this example, the control plane VCN 1116 can include the data plane mirror app tier 1140 that can include app subnet(s) 1126. The data plane mirror app tier 1140 can reside in the data plane VCN 1118, but the data plane mirror app tier 1140 may not live in the data plane VCN 1118. That is, the data plane mirror app tier 1140 may have access to the customer tenancy 1121, but the data plane mirror app tier 1140 may not exist in the data plane VCN 1118 or be owned or operated by the customer of the IaaS provider. The data plane mirror app tier 1140 may be configured to make calls to the data plane VCN 1118 but may not be configured to make calls to any entity contained in the control plane VCN 1116. The customer may desire to deploy or otherwise use resources in the data plane VCN 1118 that are provisioned in the control plane VCN 1116, and the data plane mirror app tier 1140 can facilitate the desired deployment, or other usage of resources, of the customer.

In some embodiments, the customer of the IaaS provider can apply filters to the data plane VCN 1118. In this embodiment, the customer can determine what the data plane VCN 1118 can access, and the customer may restrict access to public Internet 1154 from the data plane VCN 1118. The IaaS provider may not be able to apply filters or otherwise control access of the data plane VCN 1118 to any outside networks or databases. Applying filters and controls by the customer onto the data plane VCN 1118, contained in the customer tenancy 1121, can help isolate the data plane VCN 1118 from other customers and from public Internet 1154.

In some embodiments, cloud services 1156 can be called by the service gateway 1136 to access services that may not exist on public Internet 1154, on the control plane VCN 1116, or on the data plane VCN 1118. The connection between cloud services 1156 and the control plane VCN 1116 or the data plane VCN 1118 may not be live or continuous. Cloud services 1156 may exist on a different network owned or operated by the IaaS provider. Cloud services 1156 may be configured to receive calls from the service gateway 1136 and may be configured to not receive calls from public Internet 1154. Some cloud services 1156 may be isolated from other cloud services 1156, and the control plane VCN 1116 may be isolated from cloud services 1156 that may not be in the same region as the control plane VCN 1116. For example, the control plane VCN 1116 may be located in “Region 1,” and cloud service “Deployment 8,” may be located in Region 1 and in “Region 2.” If a call to Deployment 8 is made by the service gateway 1136 contained in the control plane VCN 1116 located in Region 1, the call may be transmitted to Deployment 8 in Region 1. In this example, the control plane VCN 1116, or Deployment 8 in Region 1, may not be communicatively coupled to, or otherwise in communication with, Deployment 8 in Region 2.

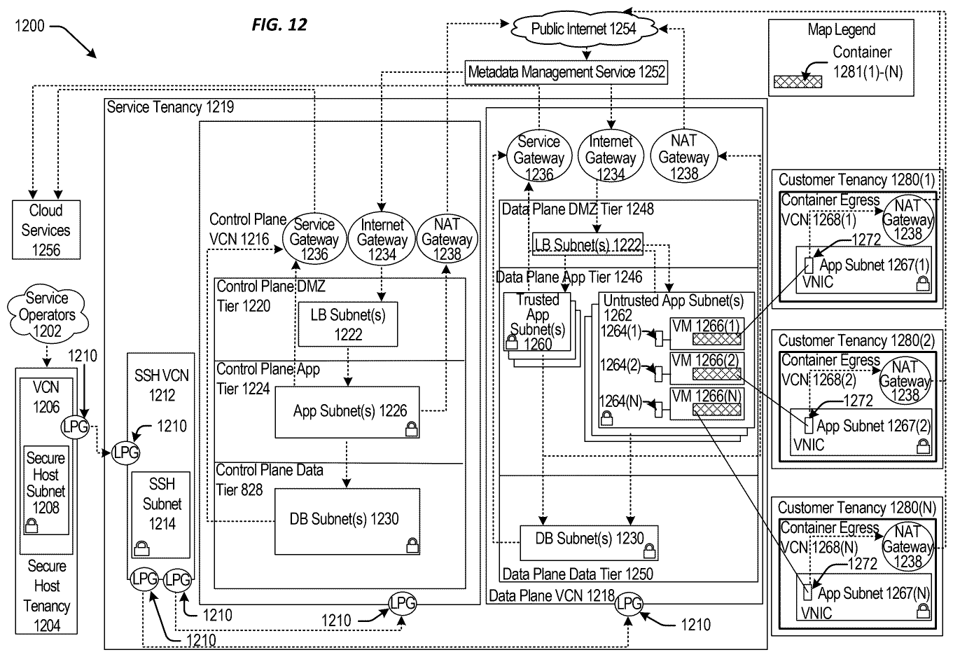

FIG. 12 is a block diagram 1200 illustrating another example pattern of an IaaS architecture, according to at least one embodiment. Service operators 1202 (e.g., service operators 1002 of FIG. 10) can be communicatively coupled to a secure host tenancy 1204 (e.g., the secure host tenancy 1004 of FIG. 10) that can include a virtual cloud network (VCN) 1206 (e.g., the VCN 1006 of FIG. 10) and a secure host subnet 1208 (e.g., the secure host subnet 1008 of FIG. 10). The VCN 1206 can include an LPG 1210 (e.g., the LPG 1010 of FIG. 10) that can be communicatively coupled to an SSH VCN 1212 (e.g., the SSH VCN 1012 of FIG. 10) via an LPG 1210 contained in the SSH VCN 1212. The SSH VCN 1212 can include an SSH subnet 1214 (e.g., the SSH subnet 1014 of FIG. 10), and the SSH VCN 1212 can be communicatively coupled to a control plane VCN 1216 (e.g., the control plane VCN 1016 of FIG. 10) via an LPG 1210 contained in the control plane VCN 1216 and to a data plane VCN 1218 (e.g., the data plane VCN 1018 of FIG. 10) via an LPG 1210 contained in the data plane VCN 1218. The control plane VCN 1216 and the data plane VCN 1218 can be contained in a service tenancy 1219 (e.g., the service tenancy 1019 of FIG. 10).

The control plane VCN 1216 can include a control plane DMZ tier 1220 (e.g., the control plane DMZ tier 1020 of FIG. 10) that can include load balancer (LB) subnet(s) 1222 (e.g., LB subnet(s) 1022 of FIG. 10), a control plane app tier 1224 (e.g., the control plane app tier 1024 of FIG. 10) that can include app subnet(s) 1226 (e.g., similar to app subnet(s) 1026 of FIG. 10), a control plane data tier 1228 (e.g., the control plane data tier 1028 of FIG. 10) that can include DB subnet(s) 1230. The LB subnet(s) 1222 contained in the control plane DMZ tier 1220 can be communicatively coupled to the app subnet(s) 1226 contained in the control plane app tier 1224 and to an Internet gateway 1234 (e.g., the Internet gateway 1034 of FIG. 10) that can be contained in the control plane VCN 1216, and the app subnet(s) 1226 can be communicatively coupled to the DB subnet(s) 1230 contained in the control plane data tier 1228 and to a service gateway 1236 (e.g., the service gateway of FIG. 10) and a network address translation (NAT) gateway 1238 (e.g., the NAT gateway 1038 of FIG. 10). The control plane VCN 1216 can include the service gateway 1236 and the NAT gateway 1238.

The data plane VCN 1218 can include a data plane app tier 1246 (e.g., the data plane app tier 1046 of FIG. 10), a data plane DMZ tier 1248 (e.g., the data plane DMZ tier 1048 of FIG. 10), and a data plane data tier 1250 (e.g., the data plane data tier 1060 of FIG. 10). The data plane DMZ tier 1248 can include LB subnet(s) 1222 that can be communicatively coupled to trusted app subnet(s) 1260 and untrusted app subnet(s) 1262 of the data plane app tier 1246 and the Internet gateway 1234 contained in the data plane VCN 1218. The trusted app subnet(s) 1260 can be communicatively coupled to the service gateway 1236 contained in the data plane VCN 1218, the NAT gateway 1238 contained in the data plane VCN 1218, and DB subnet(s) 1230 contained in the data plane data tier 1250. The untrusted app subnet(s) 1262 can be communicatively coupled to the service gateway 1236 contained in the data plane VCN 1218 and DB subnet(s) 1230 contained in the data plane data tier 1250. The data plane data tier 1250 can include DB subnet(s) 1230 that can be communicatively coupled to the service gateway 1236 contained in the data plane VCN 1218.

The untrusted app subnet(s) 1262 can include one or more primary VNICs 1264(1)-(N) that can be communicatively coupled to tenant virtual machines (VMs) 1266(1)-(N). Each tenant VM 1266(1)-(N) can be communicatively coupled to a respective app subnet 1267(1)-(N) that can be contained in respective container egress VCNs 1268(1)-(N) that can be contained in respective customer tenancies 1280(1)-(N). Respective secondary VNICs 1282(1)-(N) can facilitate communication between the untrusted app subnet(s) 1262 contained in the data plane VCN 1218 and the app subnet contained in the container egress VCNs 1268(1)-(N). Each container egress VCNs 1268(1)-(N) can include a NAT gateway 1238 that can be communicatively coupled to public Internet 1254 (e.g., public Internet 1064 of FIG. 10).

The Internet gateway 1234 contained in the control plane VCN 1216 and contained in the data plane VCN 1218 can be communicatively coupled to a metadata management service 1252 (e.g., the metadata management service 1062 of FIG. 10) that can be communicatively coupled to public Internet 1254. Public Internet 1254 can be communicatively coupled to the NAT gateway 1238 contained in the control plane VCN 1216 and contained in the data plane VCN 1218. The service gateway 1236 contained in the control plane VCN 1216 and contained in the data plane VCN 1218 can be communicatively coupled to cloud services 1256.

In some embodiments, the data plane VCN 1218 can be integrated with customer tenancies 1280. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 1246. Code to run the function may be executed in the VMs 1266(1)-(N), and the code may not be configured to run anywhere else on the data plane VCN 1218. Each VM 1266(1)-(N) may be connected to one customer tenancy 1280. Respective containers 1281(1)-(N) contained in the VMs 1266(1)-(N) may be configured to run the code. In this case, there can be a dual isolation (e.g., the containers 1281(1)-(N) running code, where the containers 1281(1)-(N) may be contained in at least the VM 1266(1)-(N) that are contained in the untrusted app subnet(s) 1262), which may help prevent incorrect or otherwise undesirable code from damaging the network of the IaaS provider or from damaging a network of a different customer. The containers 1281(1)-(N) may be communicatively coupled to the customer tenancy 1280 and may be configured to transmit or receive data from the customer tenancy 1280. The containers 1281(1)-(N) may not be configured to transmit or receive data from any other entity in the data plane VCN 1218. Upon completion of running the code, the IaaS provider may kill or otherwise dispose of the containers 1281(1)-(N).

In some embodiments, the trusted app subnet(s) 1260 may run code that may be owned or operated by the IaaS provider. In this embodiment, the trusted app subnet(s) 1260 may be communicatively coupled to the DB subnet(s) 1230 and be configured to execute CRUD operations in the DB subnet(s) 1230. The untrusted app subnet(s) 1262 may be communicatively coupled to the DB subnet(s) 1230, but in this embodiment, the untrusted app subnet(s) may be configured to execute read operations in the DB subnet(s) 1230. The containers 1281(1)-(N) that can be contained in the VM 1266(1)-(N) of each customer and that may run code from the customer may not be communicatively coupled with the DB subnet(s) 1230.

In other embodiments, the control plane VCN 1216 and the data plane VCN 1218 may not be directly communicatively coupled. In this embodiment, there may be no direct communication between the control plane VCN 1216 and the data plane VCN 1218. However, communication can occur indirectly through at least one method. An LPG 1210 may be established by the IaaS provider that can facilitate communication between the control plane VCN 1216 and the data plane VCN 1218. In another example, the control plane VCN 1216 or the data plane VCN 1218 can make a call to cloud services 1256 via the service gateway 1236. For example, a call to cloud services 1256 from the control plane VCN 1216 can include a request for a service that can communicate with the data plane VCN 1218.

FIG. 11 is a block diagram 1100 illustrating another example pattern of an IaaS architecture, according to at least one embodiment. Service operators 1102 (e.g., service operators 1002 of FIG. 10) can be communicatively coupled to a secure host tenancy 1104 (e.g., the secure host tenancy 1004 of FIG. 10) that can include a virtual cloud network (VCN) 1306 (e.g., the VCN 1006 of FIG. 10) and a secure host subnet 1308 (e.g., the secure host subnet 1008 of FIG. 10). The VCN 1306 can include an LPG 1310 (e.g., the LPG 1010 of FIG. 10) that can be communicatively coupled to an SSH VCN 1312 (e.g., the SSH VCN 1012 of FIG. 10) via an LPG 1310 contained in the SSH VCN 1312. The SSH VCN 1312 can include an SSH subnet 1314 (e.g., the SSH subnet 1014 of FIG. 10), and the SSH VCN 1312 can be communicatively coupled to a control plane VCN 1316 (e.g., the control plane VCN 1016 of FIG. 10) via an LPG 1310 contained in the control plane VCN 1316 and to a data plane VCN 1318 (e.g., the data plane VCN 1018 of FIG. 10) via an LPG 1310 contained in the data plane VCN 1318. The control plane VCN 1316 and the data plane VCN 1318 can be contained in a service tenancy 1319 (e.g., the service tenancy 1019 of FIG. 10).

The control plane VCN 1316 can include a control plane DMZ tier 1320 (e.g., the control plane DMZ tier 1020 of FIG. 10) that can include LB subnet(s) 1322 (e.g., LB subnet(s) 1022 of FIG. 10), a control plane app tier 1324 (e.g., the control plane app tier 1024 of FIG. 10) that can include app subnet(s) 1326 (e.g., app subnet(s) 1026 of FIG. 10), a control plane data tier 1328 (e.g., the control plane data tier 1028 of FIG. 10) that can include DB subnet(s) 1330 (e.g., DB subnet(s) 1130 of FIG. 11). The LB subnet(s) 1322 contained in the control plane DMZ tier 1320 can be communicatively coupled to the app subnet(s) 1326 contained in the control plane app tier 1324 and to an Internet gateway 1334 (e.g., the Internet gateway 1034 of FIG. 10) that can be contained in the control plane VCN 1316, and the app subnet(s) 1326 can be communicatively coupled to the DB subnet(s) 1330 contained in the control plane data tier 1328 and to a service gateway 1336 (e.g., the service gateway of FIG. 10) and a network address translation (NAT) gateway 1338 (e.g., the NAT gateway 1038 of FIG. 10). The control plane VCN 1316 can include the service gateway 1336 and the NAT gateway 1338.

The data plane VCN 1318 can include a data plane app tier 1346 (e.g., the data plane app tier 1046 of FIG. 10), a data plane DMZ tier 1348 (e.g., the data plane DMZ tier 1048 of FIG. 10), and a data plane data tier 1350 (e.g., the data plane data tier 1060 of FIG. 10). The data plane DMZ tier 1348 can include LB subnet(s) 1322 that can be communicatively coupled to trusted app subnet(s) 1360 (e.g., trusted app subnet(s) 1170 of FIG. 11) and untrusted app subnet(s) 1362 (e.g., untrusted app subnet(s) 1172 of FIG. 11) of the data plane app tier 1346 and the Internet gateway 1334 contained in the data plane VCN 1318. The trusted app subnet(s) 1360 can be communicatively coupled to the service gateway 1336 contained in the data plane VCN 1318, the NAT gateway 1338 contained in the data plane VCN 1318, and DB subnet(s) 1330 contained in the data plane data tier 1350. The untrusted app subnet(s) 1362 can be communicatively coupled to the service gateway 1336 contained in the data plane VCN 1318 and DB subnet(s) 1330 contained in the data plane data tier 1350. The data plane data tier 1350 can include DB subnet(s) 1330 that can be communicatively coupled to the service gateway 1336 contained in the data plane VCN 1318.

The untrusted app subnet(s) 1362 can include primary VNICs 1364(1)-(N) that can be communicatively coupled to tenant virtual machines (VMs) 1366(1)-(N) residing within the untrusted app subnet(s) 1362. Each tenant VM 1366(1)-(N) can run code in a respective container 1367(1)-(N) and be communicatively coupled to an app subnet 1326 that can be contained in a data plane app tier 1346 that can be contained in a container egress VCN 1368. Respective secondary VNICs 1372(1)-(N) can facilitate communication between the untrusted app subnet(s) 1362 contained in the data plane VCN 1318 and the app subnet contained in the container egress VCN 1368. The container egress VCN can include a NAT gateway 1338 that can be communicatively coupled to public Internet 1354 (e.g., public Internet 1064 of FIG. 10).

The Internet gateway 1334 contained in the control plane VCN 1316 and contained in the data plane VCN 1318 can be communicatively coupled to a metadata management service 1352 (e.g., the metadata management service 1062 of FIG. 10) that can be communicatively coupled to public Internet 1354. Public Internet 1354 can be communicatively coupled to the NAT gateway 1338 contained in the control plane VCN 1316 and contained in the data plane VCN 1318. The service gateway 1336 contained in the control plane VCN 1316 and contained in the data plane VCN 1318 can be communicatively coupled to cloud services 1356.

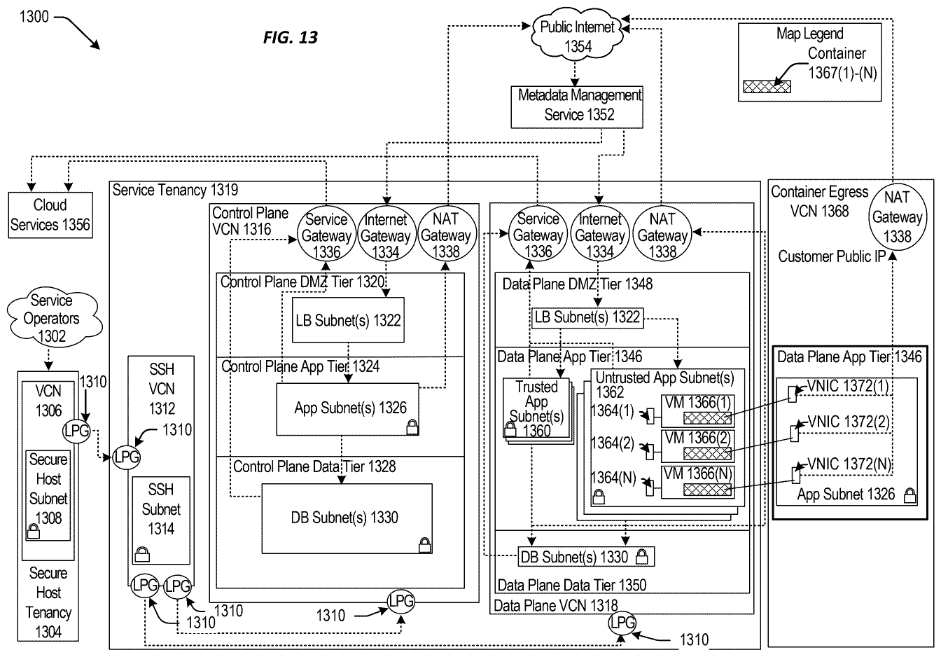

In some examples, the pattern illustrated by the architecture of block diagram 1300 of FIG. 13 may be considered an exception to the pattern illustrated by the architecture of block diagram 1100 of FIG. 11 and may be desirable for a customer of the IaaS provider if the IaaS provider cannot directly communicate with the customer (e.g., a disconnected region). The respective containers 1367(1)-(N) that are contained in the VMs 1366(1)-(N) for each customer can be accessed in real-time by the customer. The containers 1367(1)-(N) may be configured to make calls to respective secondary VNICs 1372(1)-(N) contained in app subnet(s) 1326 of the data plane app tier 1346 that can be contained in the container egress VCN 1368. The secondary VNICs 1372(1)-(N) can transmit the calls to the NAT gateway 1338 that may transmit the calls to public Internet 1354. In this example, the containers 1367(1)-(N) that can be accessed in real-time by the customer can be isolated from the control plane VCN 1316 and can be isolated from other entities contained in the data plane VCN 1318. The containers 1367(1)-(N) may also be isolated from resources from other customers.

In other examples, the customer can use the containers 1367(1)-(N) to call cloud services 1356. In this example, the customer may run code in the containers 1367(1)-(N) that requests a service from cloud services 1356. The containers 1367(1)-(N) can transmit this request to the secondary VNICs 1372(1)-(N) that can transmit the request to the NAT gateway that can transmit the request to public Internet 1354. Public Internet 1354 can transmit the request to LB subnet(s) 1322 contained in the control plane VCN 1316 via the Internet gateway 1334. In response to determining the request is valid, the LB subnet(s) can transmit the request to app subnet(s) 1326 that can transmit the request to cloud services 1356 via the service gateway 1336.

It should be appreciated that IaaS architectures 1000, 1100, 1200, 1300 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

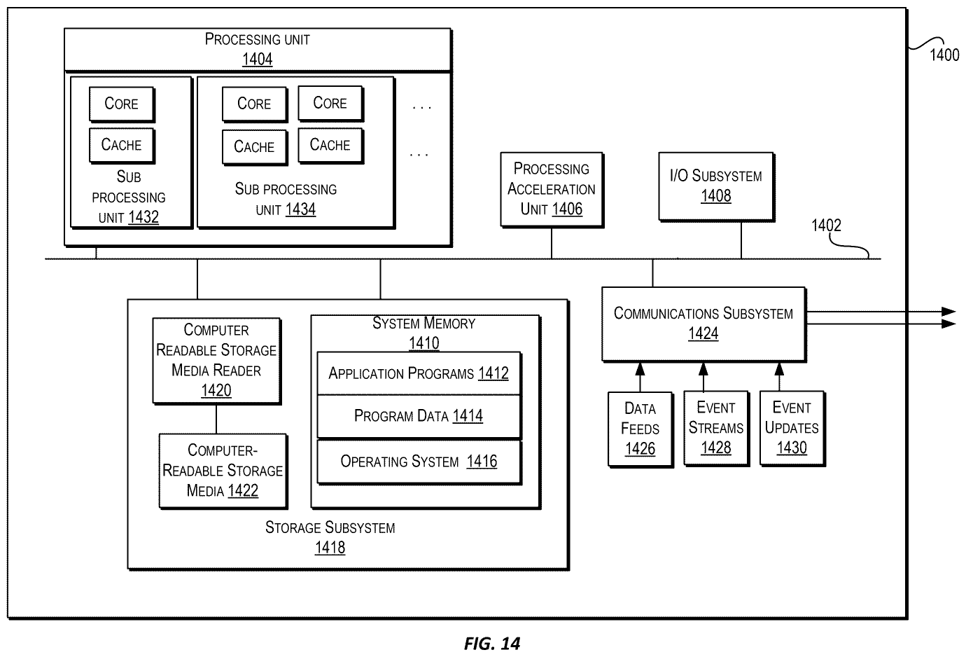

FIG. 14 illustrates an example computer system 1400, in which various embodiments may be implemented. The system 1400 may be used to implement any of the computer systems described above. As shown in the figure, computer system 1400 includes a processing unit 1404 that communicates with a number of peripheral subsystems via a bus subsystem 1402. These peripheral subsystems may include a processing acceleration unit 1406, an I/O subsystem 1408, a storage subsystem 1418 and a communications subsystem 1424. Storage subsystem 1418 includes tangible computer-readable storage media 1422 and a system memory 1410.

Bus subsystem 1402 provides a mechanism for letting the various components and subsystems of computer system 1400 communicate with each other as intended. Although bus subsystem 1402 is shown schematically as a single bus, alternative embodiments of the bus subsystem may utilize multiple buses. Bus subsystem 1402 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 1404, which can be implemented as one or more integrated circuits (e.g., a conventional microprocessor or microcontroller), controls the operation of computer system 1400. One or more processors may be included in processing unit 1404. These processors may include single core or multicore processors. In certain embodiments, processing unit 1404 may be implemented as one or more independent processing units 1432 and/or 1434 with single or multicore processors included in each processing unit. In other embodiments, processing unit 1404 may also be implemented as a quad-core processing unit formed by integrating two dual-core processors into a single chip.

In various embodiments, processing unit 1404 can execute a variety of programs in response to program code and can maintain multiple concurrently executing programs or processes. At any given time, some, or all of the program code to be executed can be resident in processing unit 1404 and/or in storage subsystem 1418. Through suitable programming, processing unit 1404 can provide various functionalities described above. Computer system 1400 may additionally include a processing acceleration unit 1406, which can include a digital signal processor (DSP), a special-purpose processor, and/or the like.

I/O subsystem 1408 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 360 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 1400 to a user or other computer. For example, user interface output devices may include, without limitation, a variety of display devices that visually convey text, graphics, and audio/video information such as monitors, printers, speakers, headphones, automotive navigation systems, plotters, voice output devices, and modems.

Computer system 1400 may comprise a storage subsystem 1418 that provides a tangible non-transitory computer-readable storage medium for storing software and data constructs that provide the functionality of the embodiments described in this disclosure. The software can include programs, code modules, instructions, scripts, etc., that when executed by one or more cores or processors of processing unit 1404 provide the functionality described above. Storage subsystem 1418 may also provide a repository for storing data used in accordance with the present disclosure.

As depicted in the example in FIG. 14, storage subsystem 1418 can include various components including a system memory 1410, computer-readable storage media 1422, and a computer readable storage media reader 1420. System memory 1410 may store program instructions 1412 that are loadable and executable by processing unit 1404. System memory 1410 may also store data 1414 that is used during the execution of the instructions and/or data that is generated during the execution of the program instructions. Various different kinds of programs may be loaded into system memory 1410 including but not limited to client applications, Web browsers, mid-tier applications, relational database management systems (RDBMS), virtual machines, containers, etc.

System memory 1410 may also store an operating system 1416. Examples of operating system 1416 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems. In certain implementations where computer system 1400 executes one or more virtual machines, the virtual machines along with their guest operating systems (GOSs) may be loaded into system memory 1410 and executed by one or more processors or cores of processing unit 1404.

System memory 1410 can come in different configurations depending upon the type of computer system 1400. For example, system memory 1410 may be volatile memory (such as random access memory (RAM)) and/or non-volatile memory (such as read-only memory (ROM), flash memory, etc.) Different types of RAM configurations may be provided including a static random access memory (SRAM), a dynamic random access memory (DRAM), and others. In some implementations, system memory 1410 may include a basic input/output system (BIOS) containing basic routines that help to transfer information between elements within computer system 1400, such as during start-up.

Computer-readable storage media 1422 may represent remote, local, fixed, and/or removable storage devices plus storage media for temporarily and/or more permanently containing, storing, computer-readable information for use by computer system 1400 including instructions executable by processing unit 1404 of computer system 1400.

Computer-readable storage media 1422 can include any appropriate media known or used in the art, including storage media and communication media, such as but not limited to, volatile and non-volatile, removable, and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media.

Byway of example, computer-readable storage media 1422 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 1422 may include, but is not limited to, Zip® drives, flash memory cards, universal serial bus (USB) flash drives, secure digital (SD) cards, DVD disks, digital video tape, and the like. Computer-readable storage media 1422 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 1400.

Machine-readable instructions executable by one or more processors or cores of processing unit 1404 may be stored on a non-transitory computer-readable storage medium. A non-transitory computer-readable storage medium can include physically tangible memory or storage devices that include volatile memory storage devices and/or non-volatile storage devices. Examples of non-transitory computer-readable storage medium include magnetic storage media (e.g., disk or tapes), optical storage media (e.g., DVDs, CDs), various types of RAM, ROM, or flash memory, hard drives, floppy drives, detachable memory drives (e.g., USB drives), or other type of storage device.

Communications subsystem 1424 provides an interface to other computer systems and networks. Communications subsystem 1424 serves as an interface for receiving data from and transmitting data to other systems from computer system 1400. For example, communications subsystem 1424 may enable computer system 1400 to connect to one or more devices via the Internet. In some embodiments communications subsystem 1424 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 602.10 family standards, or other mobile communication technologies, or any combination thereof), global positioning system (GPS) receiver components, and/or other components. In some embodiments communications subsystem 1424 can provide wired network connectivity (e.g., Ethernet) in addition to or instead of a wireless interface.

In some embodiments, communications subsystem 1424 may also receive input communication in the form of structured and/or unstructured data feeds 1426, event streams 1428, event updates 1430, and the like on behalf of one or more users who may use computer system 1400.

By way of example, communications subsystem 1424 may be configured to receive data feeds 1426 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 1424 may also be configured to receive data in the form of continuous data streams, which may include event streams 1428 of real-time events and/or event updates 1430, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1424 may also be configured to output the structured and/or unstructured data feeds 1426, event streams 1428, event updates 1430, and the like to one or more databases that may be in communication with one or more streaming data source computers coupled to computer system 1400.

Computer system 1400 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 1400 depicted in the figure is intended only as a specific example. Many other configurations that have more or fewer components than the system depicted in the figure are possible. For example, customized hardware might also be used and/or particular elements might be implemented in hardware, firmware, software (including applets), or a combination. Further, connections to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

As used herein, when an action is “based on” something, this means the action is based at least in part on at least a part of the something. As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 6, and 8 percent.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed is:

1. A method comprising:
receiving an input from an interface of a computing device associated with a user, the input comprising a query and a natural language component;
identifying, from the input, one or more key phrases associated with one or more medical concepts;
expanding the one or more medical concepts to include one or more other medical concepts that are related to the one or more medical concepts;
identifying one or more portions of a database schema associated with the one or more medical concepts and the one or more other medical concepts;
generating a prompt, wherein the prompt comprises an instruction, the one or more portions of the database schema, and an utterance associated with the natural language component;
transmitting the prompt to a machine learning model;
receiving, from the machine learning model, a query result that includes information to answer the query; and
providing the query result to the computing device associated with the user.

2. The method of claim 1, wherein expanding the one or more medical concepts comprises determining similar medical concepts identified within a medical coding system.

3. The method of claim 2, wherein the medical coding system is Systematized Nomenclature of Medicine—Clinical Terms (SNOMED CT), Prescription Normalization (RxNORM), Logical Observation Identifiers Names and Codes (LOINC), or International Classification of Diseases (ICD-10).

4. The method of claim 2, wherein determining the similar medical concepts comprises performing a search of a vector database that contains embeddings of medical concepts of the medical coding system.

5. The method of claim 2, wherein determining the similar medical concepts comprises using an embeddings-based Approximate Nearest Neighbor (ANN) algorithm.

6. The method of claim 1, wherein generating the prompt further comprises providing the one or more portions of the database schema to a second machine learning model to obtain structured object query language.

7. The method of claim 1, further comprising determining from the input an observation code that represents one or clinical findings, and wherein the prompt further comprises the observation code.

8. The method of claim 1, wherein each of the one or more medical concepts and the one or more other medical concepts comprise one or more of a medical code, a medical entity, a medical entity type associated with the medical entity, and a human-readable description.

9. A system comprising:
one or more processing systems; and
one or more computer-readable media storing instructions which, when executed by the one or more processing systems, cause the system to perform operations comprising:
receiving an input from an interface of a computing device associated with a user, the input comprising a query and a natural language component;
identifying, from the input, one or more key phrases associated with one or more medical concepts;
expanding the one or more medical concepts to include one or more other medical concepts that are related to the one or more medical concepts;
identifying one or more portions of a database schema associated with the one or more medical concepts and the one or more other medical concepts;
generating a prompt, wherein the prompt comprises an instruction, the one or more portions of the database schema, and an utterance associated with the natural language component;
transmitting the prompt to a machine learning model;
receiving, from the machine learning model, a query result that includes information to answer the query; and
providing the query result to the computing device associated with the user.

10. The system of claim 9, wherein expanding the one or more medical concepts comprises determining similar medical concepts identified within a medical coding system.

11. The system of claim 10, wherein the medical coding system is Systematized Nomenclature of Medicine—Clinical Terms (SNOMED CT), Prescription Normalization (RxNORM), Logical Observation Identifiers Names and Codes (LOINC), or International Classification of Diseases (ICD-10).

12. The system of claim 10, wherein determining the similar medical concepts comprises performing a search of a vector database that contains embeddings of medical concepts of the medical coding system.

13. The system of claim 10, wherein determining the similar medical concepts comprises using an embeddings-based Approximate Nearest Neighbor (ANN) algorithm.

14. The system of claim 9, wherein generating the prompt further comprises providing the one or more portions of the database schema to a second machine learning model to obtain structured object query language.

15. The system of claim 9, further comprising determining from the input an observation code that represents one or clinical findings, and wherein the prompt further comprises the observation code.

16. The system of claim 9, wherein each of the one or more medical concepts and the one or more other medical concepts comprise one or more of a medical code, a medical entity, a medical entity type associated with the medical entity, and a human-readable description.

17. One or more non-transitory computer-readable media storing instructions which, when executed by one or more processors, cause a system to perform operations comprising:
receiving an input from an interface of a computing device associated with a user, the input comprising a query and a natural language component;
identifying, from the input, one or more key phrases associated with one or more medical concepts;
expanding the one or more medical concepts to include one or more other medical concepts that are related to the one or more medical concepts;
identifying one or more portions of a database schema associated with the one or more medical concepts and the one or more other medical concepts;
generating a prompt, wherein the prompt comprises an instruction, the one or more portions of the database schema, and an utterance associated with the natural language component;
transmitting the prompt to a machine learning model;
receiving, from the machine learning model, a query result that includes information to answer the query; and
providing the query result to the computing device associated with the user.

18. The one or more non-transitory computer-readable media of claim 17, wherein expanding the one or more medical concepts comprises determining similar medical concepts identified within a medical coding system.

19. The one or more non-transitory computer-readable media of claim 18, wherein the medical coding system is Systematized Nomenclature of Medicine—Clinical Terms (SNOMED CT), Prescription Normalization (RxNORM), Logical Observation Identifiers Names and Codes (LOINC), or International Classification of Diseases (ICD-10).

20. The one or more non-transitory computer-readable media of claim 18, wherein determining the similar medical concepts comprises performing a search of a vector database that contains embeddings of medical concepts of the medical coding system.
